(19) United States
(12) Patent Application Publication
(10) Pub. No.: US 2022/0175860 A1
(43) Pub. Date: Jun. 9, 2022

(54) DUNALIELLA ALGA PREPARATION FOR PREVENTION AND/OR TREATMENT OF A NEURODEGENERATIVE DISEASE, A DISORDER ASSOCIATED WITH PROTEIN MISFOLDING AND COGNITIVE DECLINE

(71) Applicants: TEL HASHOMER MEDICAL RESEARCH INFRASTRUCTURE AND SERVICES LTD., Tel Hashomer (IL); NIKKEN SOHONSHA CORPORATION, Hashima-city, Gifu (JP)

(72) Inventors: Aviv SHAISH, Talmei Yehyel (IL); Dror HARATS, Ramat Gan (IL); Michal BEERI, Ra'anana (IL); Irit LUBITZ, Ramat Gan (IL)

(21) Appl. No.: 17/594,084

(22) PCT Filed: Apr. 1, 2020
(86) PCT No.: PCT/IL2020/050393

§ 371 Date: Oct. 1, 2021

Related U.S. Application Data
(60) Provisional application No. 62/827,308, filed on Apr. 1, 2019.

(51) Int. Cl. A61K 36/05; A61P 25/28

(52) U.S. Cl. CPC A61K 36/05; A61P 25/28

(57) ABSTRACT

The present invention provides neuroprotective preparations, specifically, Dunaliella alga preparations and uses thereof in the treatment and prophylaxis of neurodegenerative disorders, protein misfolding and cognitive decline.

### FIELD OF THE INVENTION

The invention relates to neuroprotective preparations. More specifically, the invention relates to Dunaliella alga preparations and uses thereof in the treatment and prophylaxis of neurodegenerative disorders, protein misfolding and cognitive decline.

### BACKGROUND ART

References considered to be relevant as background to the presently disclosed subject matter are listed below:
[1] Alzheimer's Association Report. 2018 Alzheimer's Diseases Facts and Figures. Alzheimer's & Dementia 14, 367-429.
[2] Zlokovic, B. V. Nat. Rev. Neurosci. 2011, 12: 723-738.
[3] Citron, M. et al., Nature 1992, 360(6405): 672-4.
[4] Ono, K. et al., Exp. Neurol. 2004, 189(2): 380-92.
[5] Johnson, E. J., et al. J Aging Res. 2013, 2013: 951786
[6] Bastien, J. Gene. 2004, 328: 1-16. Review.
[7] Levin, A. A. et al., Nature 1992, 355: 359-361.
[8] Akram, A. et al., Brain Res. 2010, 1318: 167-177.
[9] Selkoe, D. J. Physiol Rev. 2001, 81(2): 41-66. Review.
[10] Chakrabarti, M. et al. J. Alzheimer's. Dis. 2015, 50(2): 335-352.
[11] Ding, Y. et al., The Journal of Neuroscience, 2008, 28(45): 11622-11634.
[12] Fitz, N. F. et al. J. Neurosci. 2010, 30(20): 6862-72.
[13] WO 2018/091937.
[14] O'Hare, E. et al., Neuropharmacology. 2016, 100: 124-30.
[15] Veeraraghavalu, K. Science 2013, 340(6135): 924-c.
[16] LaClair, K. D. et al., Molecular Neurodegeneration 2013, 8:18.
[17] Grodstein, F. et al., Arch Intern Med 2007, 167(20): 2184-2190.
[18] El-Baz, F. K. and Aly, H. F., Int J Pharm Bio Sci 2016, 7(4): (B) 324-331.
[19] El-Baz, F. K. et al., Asian J of Pharmaceutical and clinical research, 2017, 10(1): 134-139.
[20] Bastien, J. Gene. 2004, 328: 1-16.
[21] Theodoulou, F. L. and Kerr, I. D. Biochem. Soc. Trans. 2015, 43(5): 1033-40.
[22] Koldamova, R. et al., Neurobiol Dis. 2014, 72 Pt A:13-21.
[23] Wahrle, S. E. et al., J Biol Chem. 2005, 280(52): 43236-42.
[24] Wahrle, S. E. et al., J Clin Invest. 2008, 118(2): 671-82.
[25] Vogelgesang, S. et al., Pharmacogenetics. 2002, 12(7): 535-41.
[26] Vogelgesang S. et al., Curr Alzheimer Res. 2004, 1(2): 121-5.
[27] Perhoc, A. B. et al., Neuropharmacology 2018, pii: S0028-3908(18)30592-6. doi: 10.1016/j.neuropharm.2018.12.018.
[28] Cohen-Kashi M. et al. Brain Res. 2009, 1284: 12-21.
[29] Reiserer, R. S. et al., Genes Brain Behav. 2007, 6(1): 54-65.
[30] Ingram, D. K. et al. Life sciences 1994, 55: 2037-2049.
[31] Moscovitch, M. et al. J Anat. 2005 207(1): 35-66.
[32] Ohno, M. et al., Neuron. 2004, 41(1): 27-33.

Acknowledgement of the above references herein is not to be inferred as meaning that these are in any way relevant to the patentability of the presently disclosed subject matter.

### BACKGROUND OF THE INVENTION

Alzheimer's disease (AD) is a progressive neurodegenerative disease that is manifested, among others, in damage and eventually destruction of brain cells, leading to memory loss and changes in cognitive and other brain functions. AD occurs mainly among the elderly population (above 65 years) and it is the most common form of dementia, accounting for 60%-80% of dementia cases [1].

The main histological features of AD are neurotic plaques (β-amyloid, Aβ), neurofibrillary tangles (tau protein), neurovascular dysfunction and also loss of neurons and synapses in the neocortex, hippocampus and other subcortical regions of the brain.

The amyloid cascade hypothesis postulates that neurodegeneration in AD is caused by abnormal accumulation of Aβ plaques in various areas of the brain. This neuritic accumulation of Aβ in the brain has been hypothesized to result from an imbalance between Aβ production and clearance [2]. In addition, a small number of AD cases (<1%), familial AD (early-onset), are linked to genetic mutations which are associated with increased Aβ production [3]. Thus, it is currently accepted that the majority of AD cases, are sporadic (late-onset) cases that may be due to faulty clearance of Aβ from the brain.

It has been suggested that retinol and β-carotene potentially inhibit amyloid β formation [4]. Hence, understanding the mechanism by which retinoids regulate amyloid β formation is of importance in developing potential therapies for the prevention and treatment of Alzheimer's disease.

Brain carotenoids have been associated with memory, which is the primary function affected in AD in several previous studies [5]. There are findings suggesting that some isomers cleaved from β-carotene affect cognition, and thus may have a role in AD, while other isomers which have no effect on cognition, interfere with or reduce the beneficial effects of the former. Retinoic acid (RA), a product of β-carotene, has two main isomers: 9-cis RA and all-trans RA, which are ligands of the retinoic X receptor (RXR) and retinoic acid receptor (RAR) [6]. While RAR (retinoic acid receptor) is activated by 9-cis RA and by all-trans RA, RXR (retinoic X receptor) is activated only by 9-cis RA [7]. RXR is a ligand-activated nuclear receptor which forms heterodimers with other nuclear receptors such as RAR, and binds to specific DNA sequences to regulate gene expression. The RXR/RAR heterodimer, expressed particularly in AD vulnerable regions [8], induces expression of brain apolipoprotein E (apoE) and cholesterol-transporters (e.g. ATP-binding cassette transporter (ABCA1) (member 1 of human transporter sub-family ABCA), also known as the cholesterol efflux regulatory protein (CERP) and ATP-binding cassette sub-family G member 1 (ABCG1)), acts as a cholesterol sensor, and decreases cellular uptake of amyloid β [9] in an apoE-dependent manner.

Thus, it is currently accepted that retinoids signaling impacts the development of AD pathology by ligand activation of RAR and RXR [10]. In APP/PS1 transgenic mice, retinoids improved AD symptoms and reduced amyloid accumulation and tau hyperphosphorylation [11].

Previous studies have shown that treatment with LXR and RXR ligands, which increase global ABCA1 expression in mice, significantly ameliorates amyloid pathology [12]. The publication WO 2018/091937 [13] also relates to RXR ligand precursors and their use in the treatment of central nervous system diseases, peripheral nervous system diseases, as well as in memory and learning impairments.

Moreover, it was demonstrated that the RXR agonist bexarotene increases amyloid β clearance via induction of ABCA1 expression and by increasing mouse apoE levels in an AD mouse model. However in spite of the exciting results demonstrated in the above bexarotene studies, it was reported that this agent had no impact on plaque burden in different other AZ animal models [14, 15]. Moreover, it has been further shown that bexarotene does not provide the expected cognitive benefit [16]. Furthermore, bexarotene was shown to be highly cytotoxic.

In addition, the publication by Grodstein, F. et al. [17] relates to a randomized trial of β-carotene supplementation and cognitive function in men, which apparently did not produce certain conclusions. Thus, the role of β-carotene in neuroprotection and its potential use in the treatment of neurodegenerative disorders is controversial.

Finally, the effect of Dunaliella salina extract in a rat model of AZ, induced by Aluminum chloride, was also described [18, 19]. The authors conclude that the biological activity of Dunaliella salina extract might be regulated by 9-cis β-carotene, protecting the brain cells from the oxidative stress in AD rats. These conclusions were based on examination of several biochemical parameters, e.g., calmodulin (CaM) level, paraoxonase 1 (PON1) activity, the antiapoptotic marker (Bcl2), brain-derived neurotrophic factor (BDNF), the generation of the DNA adducts and alteration in the expression of amyloid precursor protein, β-site APP-cleaving enzyme 1 (BACE1), and β-site APP-cleaving enzyme 2 (BACE2) in AD rats. However, the type of the AD rat model used (Aluminum chloride), and more importantly, the lack of behavioral studies warrant further investigation.

Thus, there is need in the art for effective therapeutic compositions having minimal side effects that display significant prophylactic and therapeutic effects on behavioral parameters of neurodegenerative processes, and as such, for therapeutic and prophylactic applications on neurodegenerative and protein misfolding disorders.

### SUMMARY OF THE INVENTION

By a first one of its aspects the present disclosure provides a method for preventing, treating, ameliorating, reducing or delaying the onset of at least one of a neurodegenerative disease, a disorder associated with protein misfolding, cognitive decline and any conditions or symptoms associated therewith in a subject in need thereof, comprising administering to the subject an effective amount of at least one Dunaliella algae preparation or any composition comprising thereof.

In some embodiments the method according to the present disclosure results in amelioration or reduction of at least one symptom associated with at least one of a neurodegenerative disease, a disorder associated with protein misfolding and cognitive decline in a subject in need thereof.

In other embodiments the method according to the present disclosure is wherein the symptom is at least one of short term memory impairment, long term memory impairment, impaired cognitive function, impaired learning function, β-amyloids deposition, anxiety, depression, or any combination thereof.

In further embodiments the method according to the present disclosure results in improvement of at least one of cognitive function, short term memory, long term memory, acquisition time and clearance of β-amyloids in a subject in need thereof.

In still further embodiments the method according to the present disclosure is wherein the neurodegenerative disease is at least one of Alzheimer's disease, Parkinson's disease, Mild Cognitive Impairment (MCI), Parkinson's disease with MCI, Huntington's disease, Lewy body disease, Amyotrophic lateral sclerosis (ALS), Prion disease, Motor neuron disease (MND), Spinocerebellar ataxia (SCA), Spinal muscular atrophy (SMA), Friedreich's Ataxia and any other neurodegenerative-related dementia or ataxia.

In specific embodiments the method according to the present disclosure is wherein the neurodegenerative disease is Alzheimer's disease.

In other embodiments the method according to the present disclosure is for preventing, treating, ameliorating, reducing or delaying the onset of cognitive decline.

In particular embodiments the method according to the present disclosure is wherein the Dunaliella algae is Dunaliella bardawil.

In still further specific embodiments, the method according to the present disclosure is wherein the Dunaliella algae preparation is administered orally.

By another one of its aspects the present disclosure provides at least one Dunaliella algae preparation or any composition comprising thereof for use in a method for preventing, treating, ameliorating, reducing or delaying the onset of at least one of a neurodegenerative disease, a disorder associated with protein misfolding, cognitive decline and any conditions or symptoms associated therewith in a subject in need thereof.

In some embodiments the at least one Dunaliella algae preparation or any composition comprising thereof for use according to the present disclosure is wherein the method results in amelioration or reduction of at least one symptom associated with at least one of a neurodegenerative disease, a disorder associated with protein misfolding, and cognitive decline in a subject in need thereof.

In other embodiments the at least one Dunaliella algae preparation or any composition comprising thereof for use according to the present disclosure is wherein the symptom is at least one of short term memory impairment, long term memory impairment, impaired cognitive function, impaired learning function, β-amyloids deposition, anxiety, depression or any combination thereof.

In further embodiments the at least one Dunaliella algae preparation or any composition comprising thereof for use according to the present disclosure is wherein the method results in improvement of at least one of cognitive function, short term memory, long term memory, acquisition time and clearance of β-amyloids in a subject in need thereof.

In still further embodiments, the at least one Dunaliella algae preparation or any composition comprising thereof for use according to the present disclosure is wherein the neurodegenerative disease is at least one of Alzheimer's disease, Parkinson's disease, Mild Cognitive Impairment (MCI), Parkinson's disease with MCI, Huntington's disease, Lewy body disease, Amyotrophic lateral sclerosis (ALS), Prion disease, Motor neuron disease (MND), Spinocerebellar ataxia (SCA), Spinal muscular atrophy (SMA), Friedreich's Ataxia and any other neurodegenerative-related dementia or ataxia.

In specific embodiments the at least one Dunaliella algae preparation or any composition comprising thereof for use according to the present disclosure is wherein the neurodegenerative disease is Alzheimer's disease.

In other specific embodiments, the at least one Dunaliella algae preparation or any composition comprising thereof for use according to the present disclosure is for preventing, treating, ameliorating, reducing or delaying the onset of cognitive decline.

In further specific embodiments, the at least one Dunaliella algae preparation or any composition comprising thereof for use according to the present disclosure is wherein the Dunaliella algae is Dunaliella bardawil.

In various other embodiments, the at least one Dunaliella algae preparation or any composition comprising thereof for use according to the present disclosure is wherein the Dunaliella algae preparation is administered orally.

In yet some further aspects, the invention provides a method for improving at least one of short term memory impairment, long term memory impairment, impaired cognitive function, impaired learning function, β-amyloids deposition in a subject in need thereof. In some embodiments, the method of the invention comprises the step of administering to the subject an effective amount of at least one Dunaliella algae preparation or any composition comprising thereof.

In further embodiments the method according to the present disclosure results in improvement of at least one of cognitive function, short term memory, long term memory, acquisition time and clearance of β-amyloids in a subject in need thereof.

In other embodiments the method according to the present disclosure is for preventing, treating, ameliorating, reducing or delaying the onset of cognitive decline.

In particular embodiments the method according to the present disclosure is wherein the Dunaliella algae is Dunaliella bardawil.

In still further specific embodiments, the method according to the present disclosure is wherein the Dunaliella algae preparation is administered orally.

In yet a further aspect, the present disclosure further provides at least one Dunaliella algae preparation or any composition comprising thereof for use in a method for improving at least one of short term memory impairment, long term memory impairment, impaired cognitive function, impaired learning function, β-amyloids deposition in a subject in need thereof.

By still another one of its aspects the present disclosure provides use of at least one Dunaliella algae preparation for the manufacture of a composition for preventing, treating, ameliorating, reducing or delaying the onset of at least one of a neurodegenerative disease, a disorder associated with protein misfolding, cognitive decline and any conditions or symptoms associated therewith in a subject in need thereof.

In some embodiments, the use according to the present disclosure is wherein the composition ameliorates or reduces at least one symptom associated with at least one of a neurodegenerative disease, a disorder associated with protein misfolding, and cognitive decline in a subject in need thereof.

In other embodiments, the use according to the present disclosure is wherein the symptom is at least one of short term memory impairment, long term memory impairment, impaired cognitive function, impaired learning function, β-amyloids deposition, anxiety, depression or any combination thereof.

In further embodiments, the use according to the present disclosure is wherein the composition improves at least one of cognitive function, short term memory, long term memory, acquisition time and clearance of β-amyloids in a subject in need thereof.

In still further embodiments the use according to the present disclosure is wherein the neurodegenerative disease is at least one of Alzheimer's disease, Parkinson's disease, Mild Cognitive Impairment (MCI), Parkinson's disease with MCI, Huntington's disease, Lewy body disease, Amyotrophic lateral sclerosis (ALS), Prion disease, Motor neuron disease (MND), Spinocerebellar ataxia (SCA), Spinal muscular atrophy (SMA), Friedreich's Ataxia and any other neurodegenerative-related dementia or ataxia.

In particular embodiments, the use according to the present disclosure is wherein the neurodegenerative disease is Alzheimer's disease.

In other specific embodiments, the use according to the present disclosure is for preventing, treating, ameliorating, reducing or delaying the onset of cognitive decline.

In further embodiments the use according to the present disclosure is wherein the Dunaliella algae is Dunaliella bardawil.

In still further specific embodiments, the use according to the present disclosure is wherein the Dunaliella algae preparation is administered orally.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1: Graphs showing the effect of treatment with the Dunaliella algae preparations of the invention on the weight (gr.) of wild type (WT) and Tg2576 (Tg) mice at the indicated time frame. A t-test showed significant difference between WT mice fed with the Dunaliella algae preparations of the invention (WT Duna. Prep.) and WT mice fed with regular diet (WT control) (p<0.05). Values are mean±S.E.M. Abbreviations: Tg, Tg2576; WT, wild type; Duna. Prep., Dunaliella algae preparations of the invention.

FIG. 2A-FIG. 2D: Bar graphs showing the effect of treatment with the Dunaliella algae preparations of the invention on behavioral parameters measured in WT and Tg2576 mice participating in an open field test. FIG. 2A is a bar graph representing total path. A two way ANOVA and post-hoc test showed significant effect of genotype (p<0.05); FIG. 2B is a bar graph representing percentage of cell use; FIG. 2C is a bar graph representing percentage of mouse movement time in the arena; FIG. 2D is a bar graph representing the time the mouse spent in the arena's center. A two way ANOVA showed significant effect of genotype (p<0.05). Values are mean±S.E.M. Abbreviations: Tg, Tg2576; WT, wild type; Duna. Prep., Dunaliella algae preparations of the invention.

FIG. 3: A bar graph showing the effect of Dunaliella algae preparations treatment on alternation percentage of WT and Tg2576 mice in a Y maze test (n=6-10). Values are mean±S.E.M. Abbreviations: Tg, Tg2576; WT, wild type; Duna. Prep., Dunaliella algae preparations of the invention.

FIG. 4: A bar graph showing the effect of Dunaliella algae preparations treatment on spatial learning in WT and Tg2576 mice in a Barnes maze test. Black columns represent reference memory 24 hours after the last training trial (day 1) and white columns represent long term retention 7 days after the last training trial (day 7). A two way ANOVA and post-hoc test showed significant difference (p<0.05) between Tg2576 Duna. Prep. and Tg2576 control on day 7. Values are mean±S.E.M. Abbreviations: Tg, Tg2576; WT, wild type; Duna. Prep., Dunaliella algae preparations of the invention.

FIG. 5A-FIG. 5C: Schematic drawings of the in vitro blood brain barrier (BBB) cell culture system. The model consists of a monolayer of endothelial cells, forming tight junctions that are grown on a microporous membrane filter culture insert (luminal side) and glial cells seeded at the abluminal side of the filter. Low Density Lipoprotein (LDL, 100 μl 1,600 μg/ml) from a healthy volunteer is added to the luminal side. Cells are incubated for 24 hours and then samples are collected from the luminal and abluminal side and analyzed.

FIG. 6A-FIG. 6B: FIG. 6A represents the carotenoids in the blood side of the barrier and FIG. 6B represents the carotenoids in brain side of the barrier. Human LDL was added to the luminal side (blood) and incubated for 24 hours.

FIG. 7A-FIG. 7C Bar graphs showing the liver (FIG. 7A), fat (FIG. 7B) and brain (FIG. 7C) 9-cis β-carotene and all-trans β-carotene levels in the indicated mice tissues. Values are mean±S.E. Abbreviations: 9CBC, 9-cis β-carotene; WT, wild type, βC, β-carotene.

FIG. 8A-FIG. 8B: FIG. 8A is a bar graph showing the level of insoluble beta amyloids (Aβ 42) in Tg2576 control mice and Tg2576 mice fed on Dunaliella algae preparations diet. FIG. 8B is a bar graph showing the level of soluble beta amyloids (soluble Abeta) in Tg2576 control mice and Tg2576 mice fed on Dunaliella algae preparations diet. Abbreviations: Duna. Prep., Dunaliella algae preparations of the invention; Aβ, beta amyloids.

FIG. 9A-FIG. 9C: Bar graphs showing counted RNA molecules of IL-1a (FIG. 9A), IL-1b (FIG. 9B) and TSPO (FIG. 9C) in WT and Tg2576 mice fed on Dunaliella algae preparations diet or on a control diet. Values are mean±S.E. Abbreviations: Duna. Prep., Dunaliella algae preparations of the invention.

FIG. 10: A micrograph showing the expression level of TSPO in the hippocampus of Tg2576 mice fed on Dunaliella algae preparations diet or on a control diet. T-test showed a statistical difference (p<0.05) in TSPO protein expression level between the Tg2576 mice fed on Dunaliella algae preparations diet and the control group (n=5 for both groups). Abbreviations: Duna. Prep., Dunaliella algae preparations of the invention.

FIG. 11: A bar graph showing the cholesterol and triglyceride levels in the plasma of Tg2576 mice fed on Dunaliella algae preparations diet or on a control diet as well as of wild type fed as described above. Values are mean±S.E. Abbreviations: Duna. Prep., Dunaliella algae preparations of the invention, Chol, cholesterol; TG, triglyceride.

FIG. 12: A schematic diagram showing the proposed effect of dietary 9-cis β-carotene after crossing the blood brain barrier (BBB).

FIG. 13A-FIG. 13B: High Performance Liquid Chromatography (HPLC) chromatograms of brain samples obtained from 5×FAD control mice fed on low fat chow diet (FIG. 13A) and of brain samples obtained from 5×FAD mice fed on Dunaliella algae preparation diet (FIG. 13B).

FIG. 14A-FIG. 14C: Bar graphs showing the liver (FIG. 14A), fat (FIG. 14B) and brain (FIG. 14C) of 9-cis β-carotene quantity in the indicated tissue of 5×FAD mice or wild type mice fed on Dunaliella algae preparation diet or the control diet. Values are mean±S.E. Abbreviations: Duna. Prep., Dunaliella algae preparations of the invention, 9CBC, 9-cis β-carotene, βC, β-carotene, WT, wild type, 9-cis, 9-cis β-carotene.

FIG. 15: A bar graph showing cholesterol and triglyceride levels in the plasma of 5×FAD mice and wild type mice fed on Dunaliella algae preparation diet or the control diet. Values are mean±S.E. Abbreviations: Duna. Prep., Dunaliella algae preparations of the invention, Chol, cholesterol; TG, triglyceride.

FIG. 16A-FIG. 16C: Bar graphs showing the effect of Dunaliella algae preparations treatment on short term/long term memory of 5×FAD mice. FIG. 16A is a bar graph showing new object recognition after 3 hours and FIG. 16B is a bar graph showing new object recognition after 24 hours. FIG. 16C is a bar graph showing the latency period of 5×FAD mice fed on regular diet (control) and of 5×FAD mice fed on Dunaliella algae preparation diet (Duna. Prep.). Abbreviations: Tg, Tg2576; WT, wild type; Duna. Prep., Dunaliella algae preparations of the invention.

FIG. 17: A bar graph showing recognition memory in the Y-maze test of 5×FAD mice or wild type mice fed on Dunaliella algae preparation diet or the control diet. The graph presents the mice preference index, the ratio between the time the mice spent in the new arm vs. the time they spent in both arms [PI=(time spent in new arm-time spent in old arm)/time spent in new+old arm]. A one-way ANOVA test showed significant difference (p<0.05) between 5×FAD Duna. Prep. (n=14) and 5×FAD control (n=15). Values are mean±S.E. Abbreviations: Duna. Prep., Dunaliella algae preparations of the invention.

FIG. 18: A bar graph showing the results of a Barnes Maze test of 5×FAD mice or wild type mice fed on Dunaliella algae preparation diet or the control diet. The graph presents latency time. Values are mean±S.E. Abbreviations: Duna. Prep., Dunaliella algae preparations of the invention.

FIG. 19A-FIG. 19B: Bar graphs showing the levels of insoluble (FIG. 19A) and soluble amyloid beta (FIG. 19B) in 5×FAD mice or wild type mice fed on Dunaliella algae preparation diet or the control diet. t-test showed a statistically significant difference (P<0.05) in soluble (n=3) Aβ levels extracted from 5×FAD hippocampus (B). There was no statistical difference in insoluble (n=5) Aβ levels (A). Values are mean±S.E. Abbreviations: Duna. Prep., Dunaliella algae preparations of the invention.

### DETAILED DESCRIPTION OF EMBODIMENTS

The present disclosure is based on the surprising effect of a Dunaliella powder preparation on neurodegenerative disorders such as Alzheimer's disease, D), disorders associated with protein misfolding and related conditions. More specifically, as a proof of concept, the invention demonstrated the effect of Dunaliella preparations on physiological as well as behavioral parameters that indicate cognitive function, using various AD like mouse models.

Alzheimer's disease is a devastating neurodegenerative disease accounting for 60-80 percent of dementia cases. Disease pathology worsens over time, leading to memory loss and changes in cognitive functions.

As detailed below, the Inventors have studied the effect of a Dunaliella powder preparation, on the development of AD-like neuropathology and cognitive deficits in mouse models.

The Inventors have found that 9-cis β-carotene (9βC) and all-trans BC crossed the blood brain barrier (BBB) and accumulated in the brain and that the Dunaliella powder preparation affected plasma lipid levels.

The Inventors have further found that the Dunaliella powder preparation improved long-term and short-term memory in the examined mouse models. In addition, the Dunaliella powder preparation had a positive effect on AD neuropathology and affected gene and expression of genes involved in inflammation.

As detailed herein below, the Inventors have demonstrated the effect of the Dunaliella powder preparation diet on different mouse models, having different mutations that cause Alzheimer's-like symptoms, namely Tg2576 and 5×FAD.

The Tg2576 mouse model is one of the most common transgenic mouse models used in AD studies, containing the double Swedish mutation that cause Aβ accumulation and cognitive impairment. The 5×FAD mouse model, containing 5 familial mutations in the APP and PESN genes, is characterized by rapid and aggressive Aβ accumulation and cognitive damage.

Furthermore, in order to examine the effect of the Dunaliella algae powder preparation diet on learning and memory, a battery of cognitive tests was performed, which included Y-maze spontaneous alternation, Y-maze recognition memory test, Barnes Maze and Novel Object Recognition (NOR) test design by model characterization.

The Barnes Maze test for evaluation of long-term memory and long-term retention was performed on Tg2576 mice and 5×FAD mice. The results indicate that the Dunaliella algae powder preparation significantly improved long-term memory. Long-term memory improvement was also demonstrated by the inventors.

The behavioral results show that the Dunaliella algae powder preparation significantly improved memory in almost all cognitive tests performed in different mouse models used.

Another aspect of the invention demonstrates the effect of the Dunaliella powder preparation on Aβ deposits in the mouse hippocampus. Tg2576 and 5×FAD mouse models are characterized by significant Aβ accumulation caused by their mutations. As known in the art, Alzheimer's disease brain contains soluble and insoluble Aβ. While it was initially hypothesized that the Aβ did not become toxic until the stage of insoluble, fibrillary forms of Aβ, recent studies suggest that soluble Aβ has a toxic effect on memory and strong correlation with dementia, including impair synapse structure and function and inhibited hippocampal LTP As shown by the following examples, hippocampus of treated and control transgenic mice were homogenized and their Aβ level was measured using ELISA assay. The results presented herein suggest that the Dunaliella algae powder preparation diet significantly decreased soluble Aβ level in both models, while insoluble Aβ level was significantly reduced only in Tg2576 mouse model. Still further, the Dunaliella diet had an effect on gene expression in the Tg2576 mice model as well as in the 5×FAD mice model.

More specifically, protein level expression in the hippocampus of the mouse models was examined. The emphasis was on microglia and astrocyte activation markers, TSPO and GFAP, and on synaptic plasticity presynaptic protein, synaptophysin. As shown below, in Tg2576 fed on the Dunaliella diet, a significant reduction in TSPO protein level was observed, compared to untreated mice. TSPO is a high affinity cholesterol transporter, mainly found on the outer mitochondrial membrane of steroid-synthesizing cells. Under normal conditions. TSPO has lower expression level in microglia cells. However, in response to neuroinflammation or injury, TSPO expression is upregulated, which make it an adequate microglia activation marker

Taken together, as shown in the Examples below and as indicated above, AD model mice fed with the Dunaliella preparation of the present disclosure experienced a variety of beneficial neuroprotective effects. For example, Tg2576 mice fed with the Dunaliella preparation had substantially higher survival rates as compared to Tg2576 mice fed with regular diet (Table 1). In addition, reduction in anxiety was observed in Tg2576 mice fed with Dunaliella preparation as demonstrated by an open field behavioral test (Example 2). Improvement in the learning and memory capacities of Tg2576 mice fed with the Dunaliella preparation were observed in an additional behavioral test, the Barnes maze test (Example 4). Furthermore, beneficial effect on memory for the Dunaliella preparation was also demonstrated in the 5×FAD mouse model, as shown in Example 13, which demonstrates the results obtained in a novel object recognition test.

Further to the above beneficial effects, the insoluble β-amyloid levels were significantly reduced in Tg2576 mice that were fed with the Dunaliella algae preparation over the control group (fed on regular diet, as shown in Example 7).

Therefore, in a first aspect thereof, the present disclosure provides a method for preventing, treating, ameliorating, reducing or delaying the onset of at least one of a neurodegenerative disease, a disorder associated with protein misfolding, cognitive decline and any conditions or symptoms associated therewith in a subject in need thereof. More specifically, the method of the invention may comprise the steps of administering to the subject an effective amount of at least one Dunaliella algae preparation or any composition comprising thereof.

As known in the art “Dunaliella” as herein defined is a genus of the algae family Dunaliellaceae. Dunaliella is a motile, unicellular, rod to ovoid shaped (9-11 μm) green algae, which is common in marine waters. Some Dunaliella strains can accumulate very large amounts of β-carotene, which is a pro-vitamin A (retinol and other retinoids) and a health food.

β-Carotene belongs to the carotenoids family, hydrophobic compounds which essentially consist of a C40 hydrocarbon and up to 15 double bonds. Carotenoids are classified into two types: carotenes composed of carbon and hydrogen only, such as β-carotene and lycopene; and xanthopylls, which also contain oxygen, such as lutein, canthaxanthin and zeaxanthin.

The present disclosure encompasses any Dunaliella algae known in the art and any combinations thereof, for example but not limited to Dunaliella bardawil, Dunaliella salina, Dunaliella acidophila, Dunaliella bioculata, Dunaliella lateralis, Dunaliella maritima, to name but few species known in the art.

By the term “at least one Dunaliella algae preparation” as used herein it is meant that at least one, two, three, four, five or more Dunaliella algae known in the art may be used for preparing the Dunaliella algae preparation of the present disclosure, or any combinations thereof.

As shown in the Examples below, the Inventors have demonstrated use of Dunaliella bardawil. The alga Dunaliella bardawil is a halotolerant green alga and a natural source of beta carotene (βc) which can synthesize and accumulate βc up to 10% of its dry weight. The βc formed by the alga under high light intensity and high salinity is composed of approximately 50% all-trans βc, 40% 9-cis βc and 10% α-carotene. Therefore, this alga is the best-known source for 9-cis βC in nature. It is known that synthetic 9-cis βC is extremely expensive and is not readily available for human use, since long-term toxicity trials should be performed with purified or synthetic 9-cis BC prior to approval for human use. It should be noted that further detailed preparation of Dunaliella useful in the present aspect are described in more detail herein after.

Thus, in various embodiments of the invention, the Dunaliella algae is Dunaliella bardawil. In specific embodiments the method according to the present disclosure comprises the step of administering to the subject a therapeutic effective amount of Dunaliella bardawil preparation or any composition thereof.

As known in the art, the blood brain barrier (BBB) is a highly selective barrier composed primarily of brain endothelial cells, astrocyte end-feet, pericytes, perivascular macrophages, and a basal membrane. Among the most important BBB transporters that restrict the permeability of toxins as well as therapeutic agents, are the ABC transporters [20].

ATP-binding cassette transporters (ABC transporters) are trans-membrane protein that transport a wide variety of substrates, including lipids, sterols and drugs. The transporters are localized on the surface of brain endothelial cells of the BBB and brain parenchyma. Over 20 ABC proteins representing all sub-families have been associated with several human diseases, including AD and atherosclerosis [21]. The transporter ABCA1 is transcriptionally regulated by Liver X Receptors (LXR) and Retinoic X Receptors (RXR). ABCA1 transports lipids onto apolipoproteins including apoE, which is the major cholesterol carrier in the brain and an established genetic risk factor for late-onset AD. In an AD mouse model, ABCA1 deficiency exacerbates amyloidogenesis, whereas ABCA1 overexpression diminishes amyloid load, suggesting a role for ABCA1 in Aβ metabolism [22]. Other studies have demonstrated that lack of ABCA1 increases amyloid deposition and cognitive decline in different APP transgenic mouse model accompanied by a significant decrease in the levels of soluble apoE [23]. Additionally, transgenic mice overexpressing ABCA1 in the brain have less amyloid plaques [24]. Other studies have shown that seniors with lower levels of ABCB1 in their brain epithelium had more plaque [25] and more vascular Aβ [26]. ABC transporters apparently play a major role in amyloid β clearance and therefore understanding their function and the factors regulating them may lead to the development of new therapeutic strategies against AD.

Thus, the neuroprotective effects demonstrated herein render the Dunaliella algae preparation of the present disclosure suitable for the treatment of various neurodegenerative diseases or disorders.

The term “neurodegenerative disease” as herein defined refers to a heterogeneous group of disorders that are characterized by the progressive degeneration of the structure and function of the central nervous system or the peripheral nervous system.

Any known neurodegenerative disease is encompassed by the present disclosure. For example, Alzheimer's disease, Parkinson's disease, Huntington's disease, Amyotrophic lateral sclerosis (ALS), Friedreich's Ataxia, Lewy body disease, Spinal Muscle Atrophy, motor neuron disease, synucleopathies and tauopathies.

In some embodiments the method according to the present disclosure may be specifically applicable for a neurodegenerative disease such as Alzheimer's disease, Parkinson's disease, Mild Cognitive Impairment (MCI), Parkinson's disease with MCI, Huntington's disease, Lewy body disease, Amyotrophic lateral sclerosis (ALS), Prion disease, Motor neuron disease (MND), Spinocerebellar ataxia (SCA), Spinal muscular atrophy (SMA), Friedreich's Ataxia and any other neurodegenerative-related dementia or ataxia.

As shown below, the Inventors have demonstrated (among others) a variety of beneficial effects on cognitive functions in mice models of Alzheimer's disease.

Therefore in some embodiments the method according to the present disclosure is specifically applicable for treating Alzheimer's disease.

Alzheimer's disease (AD) as known in the art relates to a progressive neurodegenerative disease that is manifested, among others, in damage and eventually destruction of brain cells, leading to memory loss and changes in cognitive and other brain functions. More specifically, AD, refers to a disorder that involves deterioration of memory and other cognitive domains that leads to death within 3 to 9 years after diagnosis. The principal risk factor for Alzheimer's disease is age. The incidence of the disease doubles every 5 years after 65 years of age, however, up to 5% of people with the disease have early onset AD (also known as younger-onset), that may appear at 40 or 50 years of age.

Many molecular lesions have been detected in Alzheimer's disease, but the overarching theme to emerge from the data is that an accumulation of misfolded proteins in the aging brain results in oxidative and inflammatory damage, which in turn leads to energy failure and synaptic dysfunction.

Alzheimer's disease may be primarily a disorder of synaptic failure. Hippocampal synapses begin to decline in patients with mild cognitive impairment (a limited cognitive deficit often preceding dementia) in whom remaining synaptic profiles show compensatory increases in size. In mild Alzheimer's disease, there is a reduction of about 25% in the presynaptic vesicle protein synaptophysin. With advancing disease, synapses are disproportionately lost relative to neurons, and this loss is the best correlate with dementia. Aging itself causes synaptic loss, which particularly affects the dentate region of the hippocampus.

Disruptions of the release of presynaptic neurotransmitters and postsynaptic glutamatereceptor ion currents occur partially as a result of endocytosis of N-methyl-D-aspartate (NMDA) surface receptors and endocytosis of α-amino-3-hydroxy-5-methyl-4-isoxazole propionic acid surface receptors. The latter further weakens synaptic activity by inducing a lasting reduction in currents after a high-frequency stimulus train. A similar shift in the balance between potentiation and depression in synapses occurs with normal aging. Intraneuronal Aβ can trigger these synaptic deficits even earlier. The normally high levels of neurotrophin receptors in cholinergic neurons in the basal forebrain are severely reduced in late-stage Alzheimer's disease. Aβ is a potent mitochondrial poison, especially affecting the synaptic pool. In Alzheimer's disease, exposure to Aβ inhibits key mitochondrial enzymes in the brain and in isolated mitochondria. Cytochrome c oxidase is specifically attacked. Consequently, electron transport, ATP production, oxygen consumption, and mitochondrial membrane potential all become impaired. The accumulation of Aβ within structurally damaged mitochondria isolated from the brains of patients with Alzheimer's disease.

There is no single linear chain of events or pathways that could initiate and drive Alzheimer's disease. AD is a progressive disease, where dementia symptoms gradually worsen over a number of years. In its early stages, memory loss is mild, but with late-stage AD, individuals lose the ability to carry on a conversation and respond to their environment. Those with AD live an average of eight years after their symptoms become noticeable to others, but survival can range from four to 20 years, depending on age and other health conditions.

The most common early symptom of AD is difficulty remembering newly learned information because AD changes typically begin in the part of the brain that affects learning. As AD advances through the brain it leads to increasingly severe symptoms, including disorientation, mood and behavior changes; deepening confusion about events, time and place; unfounded suspicions about family, friends and professional caregivers; more serious memory loss and behavior changes; and difficulty speaking, swallowing and walking.

Beside symptomatic treatments to temporarily slow the worsening of dementia symptoms, AD has no current cure, and the current treatments cannot stop AD from progressing.

Diagnosis of AD may be performed by a skilled physician and include physical examinations and diagnostic tests (for example a Mini-Mental State Exam (MMSE)). Early signs and symptoms of Alzheimer's include among others memory impairment, difficulty concentrating, planning or problem-solving, problems with finishing daily tasks, confusion, language problems such as word-finding problems or reduced vocabulary in speech or writing, changes in mood, such as depression or other behavior and personality changes.

It should be appreciated that the Dunaliella algal preparations, compositions and methods of the invention are suitable for treating any stage of AD, at any age and for any conditions and symptoms associated therewith.

As indicated above, plaques and tangles are involved with AD as well as in other age-related neurodegenerative processes. Thus, it should be appreciated that the invention further encompasses the use of the combined therapy disclosed herein for treating other age-related conditions.

With an increasingly aged population, cognitive impairment is a major health and social issue. Cognitive decline is among the most feared aspects of growing old. It is also the most costly, in terms of the financial, personal and societal burdens. It is important, because cognitive decline heralds dementia, illness and death.

As indicated above, the Inventors have shown that the AD model mice fed with the Dunaliella algae preparation of the present disclosure experienced improvement in learning and memory capacities and improvement of memory capacity, being some of the symptoms associated with various neurodegenerative diseases (e.g. AD and Huntington's disease).

Therefore in some specific embodiments the method according to the present disclosure is wherein the method results in amelioration and/or reduction of at least one symptom associated with at least one of a neurodegenerative disease, a disorder associated with protein misfolding and cognitive decline in a subject in need thereof.

By the term “amelioration” as used herein it is meant to reduce, alleviate, lighten, improve or relieve by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% at least one of the symptoms associated with at least one of a neurodegenerative disease, a disorder associated with protein misfolding and cognitive decline in a subject in need thereof.

The terms “ameliorating” and “reducing” are used herein interchangeably.

Any symptom (or any conditions or symptom) associated with at least one of a neurodegenerative disease, a disorder associated with protein misfolding and cognitive decline in a subject in need thereof is encompassed by the present disclosure. Such symptoms (or conditions) include but are not limited to decline in mental abilities (for example short-term memory and long-term memory decline, impaired learning function), behavioral and psychiatric problems, lack of coordination, unsteady gait, speech changes, tremor, confusion with time or place, decreased or poor judgment, changes in mood and personality.

In some embodiments the method according to the present disclosure is wherein the symptom is at least one of short term memory impairment, long term memory impairment, impaired cognitive function, impaired learning function, β-amyloids deposition, anxiety, depression, or any combination thereof.

In specific embodiments the symptom is herein defined is short term memory impairment. Short-term memory is a system for temporarily storing and managing information required to carry out complex cognitive tasks such as learning, reasoning, and comprehension. Short-term memory is involved in the selection, initiation, and termination of information-processing functions such as encoding, storing, and retrieving data. By the term “short term memory impairment” it is meant an impaired ability to form new episodic memories. Short term memory loss can have a substantial and negative impact on a person's quality of life. The inability to form any new episodic memories renders a person to live in a perpetual “now” state, where new events are never encoded for later recall.

In other embodiments the symptom herein defined may be long term memory impairment. The National Institutes of Health (NIH) defines “long-term memory loss” (or “long-term memory impairment”) as difficulty remembering events that occurred further in the past. Long-term memories are formed when short-term memories, or non-permanent memories, are consolidated in the hippocampus, a brain structure located in the medial temporal lobe. Once the memories are consolidated, they are available independent from the hippocampus in the neocortex, where they can be retrieved. When a patient has long-term memory loss, the patient displays problems recalling stored memories, not creating new memories.

In further embodiments the symptom herein defined may be impaired cognitive function. “Impaired cognitive function” is when a person has trouble remembering, learning new things, concentrating, or making decisions that affect their everyday life. Cognitive impairment ranges from mild to severe. With mild impairment, people may begin to notice changes in cognitive functions, but still be able to do their everyday activities. Severe levels of impairment can lead to losing the ability to understand the meaning or importance of something and the ability to talk or write, resulting in the inability to live independently.

In still further embodiments the symptom herein defined may be impaired learning function. The term “impaired learning function” as herein defined means a decrease or reduction in the learning ability which affects acquisition, organization, retention, understanding or use of verbal or nonverbal information. Impaired learning function results from impairments in one or more processes related to perceiving, thinking, remembering or learning, and may also involve difficulties with organizational skills, social perception, social interaction and perspective taking.

As indicated herein, the invention provides methods for treating, inhibiting and reducing or in other words, improving impaired cognitive functions. Impaired as used herein is meant any reduced, damaged, retarded, decreased, or attenuated cognitive parameters, for example, learning and memory functions, by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%, as compared to the cognitive functions of a healthy subject.

In still further embodiments the symptom herein defined may be β-amyloids deposition. Beta-amyloid is a protein fragment that is deposited in the brain in the form of sticky, starch-like plaques, in an increased manner in individuals with AD. Thus the term “β-amyloids deposition” means formation of beta-amyloid aggregates in the brain. Significant amyloid deposition is a characteristic feature of all patients with AD.

As shown in the following examples, Dunaliella preparations reduced both the soluble and insoluble bata amyloids. More specifically, Amyloid beta (Aβ or Abeta) denotes peptides of 36-43 amino acids that are crucially involved in Alzheimer's disease as the main component of the amyloid plaques found in the brains of people with Alzheimer's disease. The peptides derive from the amyloid precursor protein (APP), which is cleaved by beta secretase and gamma secretase to yield Aβ. Aβ molecules can aggregate to form flexible soluble oligomers which may exist in several forms. It is now believed that certain misfolded oligomers (known as “seeds”) can induce other Aβ molecules to also take the misfolded oligomeric form that is toxic to nerve cells. More specifically, Aβ is a 4 kDa peptide (with 40- and 42-amino acid residue peptides as the predominant species) derived from proteolytic cleavage of a precursor protein termed amyloid precursor protein. Aβ monomers readily aggregate in aqueous medium, giving rise to various types of assemblies including oligomers, protofibrils and amyloid fibrils. While AβOs are soluble and may spread throughout the brain, amyloid fibrils are larger and insoluble, and assemble into amyloid plaques, forming histological lesions that are characteristic of AD.

The insoluble fibrillar beta amyloid lesions (known as neuritic plaques) do not necessarily correlate very well with disease progression, suggesting that are not directly causal. However, the soluble form of amyloid seems to better correlate with disease progression. It appears that high MW weight oligomers may cause synaptic loss and, ultimately, memory loss in AD.

However, it is also present in many normal adults and observed in individuals with Mild Cognitive Impairment (MCI).

The invention therefore in certain embodiments thereof, provides methods for treating, preventing, inhibiting, reducing, eliminating, protecting or delaying the onset of age-associated mild cognitive impairment (MCI).

“Age-associated mild cognitive impairment (MCI)”, as used herein is a condition that causes cognitive changes. MCI that primarily affects memory may be classified as “amnestic MCI” where the subjects experience impairment in memorizing information that relate to recent events, appointments, conversations or recent events. MCI that affects thinking skills other than memory is known as “nonamnestic MCI”. Thinking skills that may be affected by nonamnestic MCI include the ability to make sound decisions, judge the time or sequence of steps needed to complete a complex task, or visual perception.

Normal aging is associated with a decline in various memory abilities in many cognitive tasks; the phenomenon is known as age-related memory impairment (AMI), age-associated memory impairment (AAMI) or age-associated cognitive decline (ACD). The ability to encode new memories of events or facts and working memory shows decline in both cross-sectional and longitudinal studies. Studies comparing the effects of aging on episodic memory, semantic memory, short-term memory and priming find that episodic memory is especially impaired in normal aging; some types of short-term memory are also impaired. The deficits may be related to impairments seen in the ability to refresh recently processed information.

Normally, there is little age-associated decline in some mental functions such as verbal ability, some numerical abilities and general knowledge but other mental capabilities decline from middle age onwards, or even earlier. The latter include aspects of memory, executive functions, processing speed and reasoning. It should be therefore appreciated that in some embodiments, the invention provides combined treatment for any cognitive decline, specifically cognitive decline associated with age, specifically, the age of 50, 55, 60, 65, 70, 75, 80, 85, 90, 95 and more, years of age.

In other embodiments the symptom or condition as herein defined is anxiety. By the term “anxiety” it is meant an abnormal and overwhelming sense of apprehension and fear often marked by physical signs (such as tension, sweating, and increased pulse rate), by doubt concerning the reality and nature of the threat, and by self-doubt about one's capacity to cope with it.

In further embodiments the symptom or condition as herein defined is depression. As known in the art the term “depression” means a state of low mood and aversion to activity that can affect a person's thoughts, behavior, tendencies, feelings, and sense of well-being. A depressed mood is a normal temporary reaction to life events—such as loss of a loved one. It is also a symptom of some physical diseases and a side effect of some drugs and medical treatments. Depressed mood may also be a symptom of some mood disorders such as major depressive disorder or dysthymia.

As indicated above the Inventors have shown that AD model mice fed with the Dunaliella algae preparation of the present disclosure experienced, inter alia, reduction in anxiety and improvement in the learning and memory capacities. In addition the Inventors have shown that in the AD model mice fed with the Dunaliella algae preparation of the present disclosure the level of insoluble β-amyloid was significantly reduced as compared to the control group (fed with regular diet).

Therefore in some embodiments the method according to the present disclosure is wherein said method results in improvement of at least one of cognitive function, short term memory, long term memory, acquisition time and clearance of β-amyloids in a subject in need thereof.

By the term “improvement” as used herein it is meant any recovery, advance or enhancement by at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100% of at least one of cognitive function, short term memory, long term memory, acquisition time and clearance of β-amyloids in a subject in need thereof, specifically, as compared with any of the behavioral parameters examined, specifically, long term memory, short term memory, cognitive function, learning function, anxiety and depression, determined before treatment of the subject with the Dunaliella preparations of the invention.

It should be further understood that in some embodiments, the present invention further provides improvement of cognitive function as discussed above, specifically, learning function, short term and long term memory, and reduction in anxiety and depression, for subjects that do not necessarily suffer from neurodegenerative disorders, specifically, the invention provides methods for improving cognitive function for healthy subjects.

In further particular embodiments of the present disclosure, the term “improvement” as used herein it is meant any recovery, advance or enhancement as indicated above of at least one of short term memory impairment, long term memory impairment, impaired cognitive function, impaired learning function, β-amyloids deposition in a subject in need thereof.

The method, Dunaliella algae preparation for use and compositions and uses as herein defined are also useful for the preventing, reducing or treating cognitive decline.

Therefore in some embodiments the method according to the present disclosure is for preventing, treating, ameliorating, reducing or delaying the onset of cognitive decline.

By the term “cognitive decline” as used herein it is meant impairment of cognitive function of an individual to the point where normal functioning is impossible without treatment. Some of the common signs of cognitive decline are confusion, poor motor coordination, loss of short-term or long-term memory, identity confusion and impaired judgement.

As shown below, the Inventors have demonstrated that the insoluble β-amyloid levels were significantly reduced in Tg2576 mice that were fed with the Dunaliella algae preparation of the present disclosure over the control group (on regular diet). As known in the art, various human degenerative conditions, including Alzheimer's disease, light-chain amyloidosis and the spongiform encephalopathies, are associated with the deposition in tissue of proteinaceous aggregates (which are misfolded proteins) known as “amyloid fibrils” or “plaques”.

Therefore in some embodiments the methods, uses and Dunaliella algae preparation for use as herein defined are for preventing, treating, ameliorating, reducing or delaying the onset of a disorder associated with protein misfolding comprising administering to said subject an effective amount of at least one Dunaliella algae preparation or any composition comprising thereof.

“Protein misfolding and aggregation” as used herein, relates to an impaired physical process by which a protein chain acquires its native three-dimensional structure, a conformation that is usually biologically functional, in an expeditious and reproducible manner. Protein folding is the physical process by which a polypeptide folds into its characteristic and functional three-dimensional structure from random coil. Each protein exists as an unfolded polypeptide or random coil when translated from a sequence of mRNA to a linear chain of amino acids. Amino acids interact with each other to produce a well-defined three-dimensional structure, the folded protein, known as the native state. The correct three-dimensional structure is essential to function, although some parts of functional proteins may remain unfolded. Failure to fold into native structure generally produces inactive proteins, but in some instances misfolded proteins have modified or toxic functionality. Several neurodegenerative and other diseases are believed to result from the accumulation of amyloid fibrils formed by misfolded proteins.

More specifically, under some conditions, proteins may not fold into their biochemically functional forms resulting in protein denaturation. A fully denatured protein lacks both tertiary and secondary structure, and exists as a so-called random coil. Under certain conditions some proteins can refold; however, in many cases, denaturation is irreversible. Cells may protect their proteins against the denaturing influence of heat with enzymes known as chaperones or heat shock proteins, which assist other proteins both in folding and in remaining folded. Some proteins never fold in cells at all except with the assistance of chaperone molecules, which either isolate individual proteins so that their folding is not interrupted by interactions with other proteins or help to unfold misfolded proteins, giving them a second chance to refold properly. This function is crucial to prevent the risk of precipitation into insoluble amorphous aggregates.

It is currently known that trace amounts of aggregates of a variety of proteins might occur spontaneously, particularly during ageing, and that such aggregates could account for subtle impairments of cellular function even in the absence of an evident amyloid phenotype. The general pattern of disorders associated with protein misfolding is abnormal tendency of proteins to aggregate as a result of misfolding.

Thus by the term “disorder associated with protein misfolding” as used herein it is referred to a disease or disorder directly or indirectly resulting from accumulation of misfolded aggregates of proteins in organs or tissues. More specifically, aggregated proteins are associated with prion-related illnesses such as Creutzfeldt-Jakob disease, bovine spongiform encephalopathy (mad cow disease), amyloid-related illnesses such as Alzheimer's disease and familial amyloid cardiomyopathy or polyneuropathy, as well as intracytoplasmic aggregation diseases such as Huntington's and Parkinson's disease. These age onset degenerative diseases are associated with the aggregation of misfolded proteins into insoluble, extracellular aggregates and/or intracellular inclusions including cross-beta sheet amyloid fibrils. It is not completely clear whether the aggregates are the cause or merely a reflection of the loss of protein homeostasis, the balance between synthesis, folding, aggregation and protein turnover. Misfolding and excessive degradation instead of folding and function leads to a number of proteopathy diseases such as antitrypsin-associated emphysema, cystic fibrosis and the lysosomal storage diseases, where loss of function is the origin of the disorder.

Also encompassed by the term “disorder associated with protein misfolding” is a group of disorders associated with beta-amyloid protein aggregation that includes Alzheimer's disease (AD), where deposits of a protein precursor called beta-amyloid build up (termed plaques) in the spaces between nerve cells and twisted fibers of tau protein build up (termed tangles) inside the cells.

More specifically, “Beta-amyloid protein aggregations” as used herein relates to cerebral plaques laden with β-amyloid peptide (Aβ) and dystrophic neurites in neocortical terminal fields as well as prominent neurofibrillary tangles in medial temporal-lobe structures, which are important pathological features of Alzheimer's disease. Subsequently, loss of neurons and white matter, congophilic (amyloid) angiopathy are also present.

Aβ peptides are natural products of metabolism consisting of 36 to 43 amino acids. Monomers of Aβ40 are much more prevalent than the aggregation-prone and damaging Aβ42 species. β-amyloid peptides originate from proteolysis of the amyloid precursor protein by the sequential enzymatic actions of beta-site amyloid precursor protein-cleaving enzyme 1 (BACE-1), a β-secretase, and γ-secretase, a protein complex with presenilin 1 at its catalytic core. An imbalance between production and clearance, and aggregation of peptides, causes Aβ to accumulate, and this excess may be the initiating factor in Alzheimer's disease.

β-amyloid can also grow into fibrils, which arrange themselves into β-pleated sheets to form the insoluble fibers of advanced amyloid plaques. Soluble oligomers and intermediate amyloid are the most neurotoxic forms of Aβ. In brain-slice preparations, dimers and trimers of Aβ are toxic to synapses. Experimental evidence indicates that Aβ accumulation precedes and drives tau protein aggregation.

“Tau protein” as used herein, refers to neurofibrillary tangles, which are filamentous inclusions in pyramidal neurons, characteristic for Alzheimer's disease and other neurodegenerative disorders termed tauopathies. Elucidation of the mechanisms of their formation may provide targets for future therapies. Accumulation of hyperphosphorylated Tau protein as paired helical filaments in pyramidal neurons is a major hallmark of Alzheimer disease (AD). Besides hyperphosphorylation, other modifications of the Tau protein, such as cross-linking, are likely to contribute to the characteristic features of paired helical filaments, including their insolubility and resistance against proteolytic degradation. These neurofibrillary tangles, consist of hyperphosphorylated and aggregated forms of the microtubule-associated protein tau.

Under non-pathological conditions, tau is a developmentally regulated phosphoprotein that promotes assembly and stability of microtubules and is thus involved in axonal transport. In AD and other tauopathies, tau proteins aggregate and form fibrillar insoluble intracellular inclusions, so-called neurofibrillary tangles. It has been suggested that ionic interactions and covalent cross-linking contribute to pathological Tau aggregation and tangle formation. Reactive carbonyl compounds, which are increased under conditions of oxidative stress and in aging have been proposed as potential compounds responsible for tau aggregation.

It should be noted that the methods of the invention may be also applicable in some embodiments thereof for treating synucleopathies. “Alpha-synuclein pathology disorders” as used herein are disorders characterized by the presence of a specific intracellular protein aggregates (inclusion bodies) known as Lewy bodies that contain mainly alpha-synuclein protein. Alpha-synuclein protein consists of 140 amino acids and is found naturally as an unfolded cytoplasmic protein in neuronal synaptic areas.

Overexpression of alpha-synuclein interrupts normal cell functions and leads to decreases in neurite outgrowth and cell adhesion. Alpha-synuclein aggregates comprising monomeric, oligomeric intermediate, or fibrillar forms are thought to be involved in a critical step in the pathogenesis of Parkinson's disease (PD) and in other alpha-synucleinopathies, such as multiple system atrophy (MSA) and dementia with Lewy bodies (DLB). These chronic neurodegenerative diseases of the CNS are characterized by the development of Lewy bodies containing alpha-synuclein protein. Oligomeric and monomeric alpha-synuclein have both been detected in cerebrospinal fluid and plasma samples from PD patients, suggesting that small aggregates of alpha-synuclein access the extracellular space.

Still further, as noted herein above, the invention provides Dunaliella algal preparations, compositions, kits and methods applicable in protecting against any neurodegeneration, or any neuronal damage as discussed herein. Neurodegeneration is a common theme of many nervous system diseases and disorders, such as Parkinson's disease, Alzheimer's disease, ALS, head trauma and epilepsy.

A common theme of these diseases and disorders is the loss of neural cell functions and/or neural cell death or damage. Here, the Inventors disclose Dunaliella algal preparations, composition and methods involving exposing neural cells to Dunaliella algal preparations, whether directly or through administration to a patient, for neuro-protection or protection from any neuronal damage or injury and thereby prevention and treatment of pathologies which cause neural cell function deterioration and death.

When referring to cell damage, the term “damage” or injury relates to any disruption of physiological cell functions or cell death. Non-limiting examples for disruption of physiological cell functions include: oxidative stress (for example, lipid peroxidation, DNA and RNA oxidation and protein oxidation), non-specific glycation, protein misfolding, DNA mutation, loss of any cellular structure integrity, metabolic stress, ionizing and non-ionizing radiation damage and chemical stress (for example, exposure to acid or basic substances).

Accordingly, the expression “protection from neural cell function deterioration and death” means either preventing or decreasing neural death, or preventing or decreasing the deterioration in neural function (as exemplified for instance by secretion of neurotransmitters, dendrite and axonal growth, transfer of electrical impulses, response to stimuli, maintaining structural integrity of myelin sheaths and Ranvier's nodes, etc.)

The term “neural cell function” relates to any normal physiological cellular activity, depending on the specific cell type. Non-limiting examples of such functions include cell viability, secretion of neurotransmitters, dendrite and axonal growth, transfer of electrical impulses and response to stimuli in neurons, maintaining structural integrity of myelin sheaths and Ranvier's nodes in oligodendrocytes and Schwann cells, and supplying nutrients and oxygen, and recycling neurotransmitters in astrocytes.

It should be appreciated that throughout this specification, the term “neural cell” relates to cells that may be any one of central nervous system neurons and glial cells, astrocyte, neuron cells, oligodendrocyte, Schwann cells, satellite cells, spindle cells, neuronauditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells of eye, photoreceptor green-sensitive cone cells of eye, photoreceptor red-sensitive cone cells of eye, proprioceptive primary sensory neurons, touch-sensitive primary sensory neurons, type I carotid body cells, type II carotid body cells, type I hair cells of vestibular apparatus of ear, type II hair cells of vestibular apparatus of ear, type I taste bud cells, autonomic neuron cells, cholinergic neural cells, adrenergic neural cells, peptidergic neural cells, sense organ and peripheral neuron supporting cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells and enteric glial cells.

Since the invention provides Dunaliella algal preparations, compositions and methods for protection from, reduction, prevention or inhibition of deterioration in neural cell function in a subject in need thereof, it is important to clearly define the scope of the term “neural cell function”. Herein, this term relates to any normal physiological cellular activity, depending on the specific cell type. Non-limiting examples of such functions include cell viability, secretion of neurotransmitters, dendrite and axonal growth, transfer of electrical impulses and response to stimuli in neurons, maintaining structural integrity of myelin sheaths and Ranvier's nodes in oligodendrocytes and Schwann cells, and supplying nutrients and oxygen, and recycling neurotransmitters in astrocytes.

As disclosed herein above, neurodegeneration is the umbrella term for the progressive loss of structure or function of neurons, including death of neurons. Many neurodegenerative diseases including Parkinson's, Alzheimer's, ALS and Huntington's occur as a result of neurodegenerative processes. Other examples of neurodegeneration include Friedreich's ataxia, Lewy body disease, spinal muscular atrophy, multiple sclerosis, frontotemporal dementia, corticobasal degeneration, progressive supranuclear palsy, multiple system atrophy, hereditary spastic paraparesis, amyloidoses and Charcot Marie Tooth. It should not be overlooked that normal aging processes include progressive neurodegeneration.

Still further, it should be appreciated that the invention provides methods for treating or preventing any neuro-pathological condition. The term “neuro-pathological condition” relates to any pathological condition caused by, or which causes, or is associated with neural cell disorders, such as any deterioration of the neural cell functions or viability. As explained herein, such conditions may be neurodegenerative disorders, brain traumas, metabolic disorders which affect the nervous system, such as phenylketonuria, immunological disorders which affect the brain, such as Hashimoto's Thyroiditis, genetic diseases which affects neural cells, such as Tay-Sachs disease, metachromatic leukodystrophy, Krabbe disease, Fabry disease, Gaucher disease, Farber disease, and Niemann-Pick disease, nutrient deficiencies such as vitamin B₆ and D deficiencies, and any sequelae which affects the nervous system.

It should be further appreciated that the Dunaliella algal preparations, methods and compositions of the invention may be applicable for treating neuro-pathological and neurodegenerative disorders or any pathologic condition associated therewith. It is understood that the interchangeably used terms “associated”, linked” and “related”, when referring to pathologies herein described, mean diseases, disorders, conditions, or any pathologies which at least one of: share causalities, co-exist at a higher than coincidental frequency, or where at least one disease, disorder condition or pathology causes the second disease, disorder, condition or pathology. Such conditions may include for example, Parkinson's disease, Alzheimer's disease, amyotrophic lateral sclerosis, head trauma, epilepsy, stroke, neuromyotonia/Isaacs syndrome, lower motor neuron lesion, Werdnig-Hoffman disease, amyotrophic lateral sclerosis, Kennedy disease, organophosphate poisoning, benzodiazepine withdrawal, magnesium deficiency, myalgic encephalomyelitis, dehydration, fatigue, lyme disease, myasthenia gravis, rabies, fibromyalgia, subarachnoid hemorrhage, intracerebral hemorrhage, occlusion and stenosis of precerebral arteries, occlusion and stenosis of basilar artery, occlusion and stenosis of carotid artery, occlusion and stenosis of vertebral artery, occlusion of cerebral arteries, cerebral thrombosis with or without cerebral infarction, cerebral embolism with or without cerebral infarction, transient cerebral ischemia, basilar artery syndrome, vertebral artery syndrome, subclavian steal syndrome, vertebrobasilar artery syndrome, transient ischemic attack (TIA), cerebral atherosclerosis, hypertensive encephalopathy, cerebral aneurysm, cerebral arteritis, Moyamoya Disease, nonpyogenic thrombosis of intracranial venous sinus, atherosclerosis, atherosclerosis of renal artery, atherosclerosis of native arteries of the extremities, intermittent claudication, aortic aneurysm, dissection of aorta, dissection of carotid artery, dissection of iliac artery, dissection of renal artery, dissection of vertebral artery, erythromelalgia, and polyarteritis nodosa.

The terms “disease”, “disorder” or “condition” refer to a state in which there is a disturbance of normal functioning. By the term “at least one” in the context of the disease”, “disorder” or “condition” as herein defined it is meant that a beneficial (therapeutic) effect may be achieved in at least one, for example 2, 3, 4, 5 or more diseases, disorders or conditions as herein defined, by the Dunaliella algae preparation of the present disclosure.

The terms “treat, treating, treatment” as used herein mean ameliorating or reducing one or more clinical indicia of disease activity in a subject having a disease or disorder as herein defined. Amelioration or reduction in the clinical indicia of disease may be subtle or significant.

Methods for preventing or delaying the onset of at least one of a neurodegenerative disease, a disorder associated with protein misfolding, cognitive decline and any conditions or symptoms associated therewith in a subject in need thereof comprising administering to said subject an effective amount of at least one Dunaliella algae preparation or any composition comprising thereof are also encompassed by the present disclosure.

By the term “preventing” it is meant to provide a “preventive treatment” or “prophylactic treatment”, namely acting in a protective manner, defending against or preventing something, especially a condition or disease as herein defined.

The disease, disorder or condition as herein defined often begin subtly but progress until they significantly impede the affected individual's quality of life. Factors such as age, genetics and lack of proper nutrients contribute to the development of the disease. By the term “delaying the onset” in the context of the disorder, disease or condition as defined herein, it is meant any postponement, suspension, impediment or retardation of the manifestation of the disease or symptoms associated therewith as herein defined.

“Subject in need thereof” as used herein means warm-blooded animals (such as for example humans, rats, mice, dogs, cats, guinea pigs and primates). In some embodiments the subject is diagnosed with the disease, disorder or condition herein defined. Diagnosis of the disease, disorder or condition herein defined may be performed by a skilled physician, as known in the art.

The “Dunaliella algae preparation” or “Dunaliella preparation” of the present disclosure may be prepared by any known method. In some embodiments the Dunaliella algae preparation is prepared as described by the Examples below and is a Dunaliella algae powder preparation.

In some embodiments the Dunaliella preparation of the present disclosure is prepared as an extract. By the term “extract” it is meant any substance or a mixture of substances extracted from Dunaliella, using enzymes, organic solvents or hydrophilic solvents for extraction. In other words, the term extract encompasses substances obtained by using either organic solvents such as, for example, alcohols (e.g. ethanol), hexane, ethyl-acetate or isopropyl-alcohol, or by hydrophilic solvents such as water. Alternatively, an extract may be prepared by any physical extraction such as cutting, mincing, grinding, either fresh, frozen or dried Dunaliella material. The extracts may be dried after said extraction and may be further processed (extracted) by any extraction method, independently from previous extraction steps. Such steps may be repeated independently. Furthermore, other extraction techniques may be employed, non-limiting examples of which include chromatography, including size-exclusion, hydrophobic interaction, and anion and cation exchangers, differential centrifugation, differential precipitation (for example, using ammonium sulfate), differential filtration and dialysis.

As indicated above, in some embodiments, fresh, frozen, dried or evaporated Dunaliella material may be used for any of the above preparation procedures.

In some embodiments the Dunaliella preparation of the present disclosure is a powder preparation.

The “powder preparation” as used herein may be prepared by any method known in the art. In some embodiments, the powder preparation is as disclosed by U.S. Pat. No. 8,722,057. More specifically, different methods of preparation will produce powders with different properties. In order to prepare powders consisting of particles having a particular size and shape, careful selection of the preparation technique is necessary. Grinding, the thermal decomposition of solids and the deposition of solids from the liquid or vapor phase are the commonest techniques used for the preparation of powders. Any pharmaceutically compatible binding agents, excipients and/or adjuvant materials can be included as part of the powder preparation as herein defined.

A raw material to be used for carrying out a method for producing the present invention is microalgae, and preferably the algae belonging to the genus Dunaliella as one type of the green algae. The algae belonging to the genus Dunaliella are known to produce and store a large amount of β-carotene in the alga body. In particular, since Dunaliella bardawil and Dunaliella salina store a large amount of β-carotene in the alga bodies, they are further preferable to be used.

The algae belonging to the genus Dunaliella are cultured in a culture device such as a culture tank and a culture pool outside or inside for a predetermined time, and then pumped out from such a culture facility by using a pumping means such as a pump. The culture solution pumped out is filtered through a predetermined mesh net so as to remove foreign substances contaminated in the culture device.

The culture solution from which foreign substances are removed is dehydrated by a centrifuge so that the solid part in the culture solution is concentrated to a predetermined concentration. The concentration of the solid part in the culture solution after centrifugation is preferably 10 to 30% by weight from the viewpoint that the culture solution has fluidity although it is concentrated. Note here that the centrifuge is preferably an apparatus capable of carrying out centrifugation of the culture solution in a batch or continuous manner, and more preferably an apparatus capable of carrying out centrifugation continuously from the viewpoint of workability and productivity. Furthermore, as a centrifuge, generally available centrifuges are used, and the rotation rate of a rotor of the centrifuge is not particularly limited but it is set for each centrifuge used so as to have the above-mentioned concentration of the solid part of the culture solution.

In the present invention, a pH adjusting step is carried out in which a culture solution concentrated to a predetermined concentration is treated in a basic state. In the pH adjusting step, a basic compound, its aqueous solution or the like is added to the culture solution concentrated to a predetermined concentration, and the culture solution is preferably stirred and mixed with a stirring device such as a stirrer in a highly basic state in which the hydrogen ion exponent, i.e., pH is 9.5 or higher at a temperature of about 25° C., more preferably stirred and mixed in a highly basic state in which pH is 10.0 or higher, and most preferably stirred and mixed in a highly basic state in which pH is 11.0 or higher. It is not preferable that pH is less than 9.5 because it is difficult to stably control Dunaliella powder so as to have a total pheophorbide amount of 160 mg % or less and an existing pheophorbide amount of 100 mg % or less throughout the year.

In general, in the production step of Dunaliella powder, various steps are usually carried out in neutral to weak basic states. If pH adjusting treatment is added as in the present invention, not only another step is added, but also a neutralization treatment step is carried out if necessary as mentioned below. For such reasons, productivity and the like may be affected. Therefore, conventionally, an idea of allowing a culture solution concentrated to a predetermined concentration to be in a strong basic has not been reached.

Preferable examples of basic compounds to be used in the pH adjusting step include lithium hydroxide, sodium hydroxide, potassium hydroxide, rubidium hydroxide, cesium hydroxide, tetramethylammonium hydroxide, calcium hydroxide, strontium hydroxide, barium hydroxide, thallium hydroxide, and guanidine. More preferable examples include widely used sodium hydroxide, potassium hydroxide, and calcium hydroxide. Furthermore, two or more thereof may be used together. In addition, an aqueous solution thereof having an arbitrary concentration can be used.

If necessary, after the pH adjusting treatment is carried out, a neutralization treatment step is carried out in order that the liquid property is made to be in a neutral range around pH 7 at a temperature of 25° C. This step will be necessary in the case where it is difficult to distribute Dunaliella powder at such a high pH when, for example, the obtained Dunaliella powder is sold as health foods or processed foods. Note here that if another neutralization treatment step is carried out when processed foods are produced using the Dunaliella powder, the neutralization treatment step is not necessarily carried out in the present invention.

As compounds to be used in the neutralization treatment step, an inorganic acid or an organic acid is used. Examples of the inorganic acid include hydrochloric acid, phosphoric acid, sulfuric acid, and nitric acid. Examples of the organic acid include formic acid, acetic acid, citric acid, and oxalic acid. Furthermore, two or more thereof may be used together. In addition, aqueous solutions thereof having an arbitrary concentration can be used.

Then, for removing dissolved salt or precipitated salt contained in the Dunaliella culture solution as well as salt generated in the neutralization treatment step, a desalting treatment step can be carried out. For the desalting treatment step, well-known methods can be used. For example, desalting treatment using a chitosan solution, which is described in Japanese Patent Laid-Open No. 1995-000147, can be used.

Then, in the present invention, for further decreasing pheophorbide harmful to a human body, which is contained in a culture solution subjected to desalting treatment and concentrated to a predetermined concentration, or for killing general bacteria, a heat treatment step may be carried out at a predetermined temperature for a predetermined time. By combining it with the above-mentioned pH adjusting treatment step, various pheophorbide amounts can be decreased more effectively. The heat treatment step is carried out preferably in a temperature range from 70° C. to 140° C., and more preferably in a temperature range from 80° C. to 130° C. It is not preferable that heat treatment temperature is carried out at a temperature of less than 70° C. because it takes a long time to decrease various pheophorbide amounts or carry out sterilization and the content of β-carotene in Dunaliella powder is lowered due to oxidative degradation. Furthermore, it is not preferable that heat treatment is carried out at a temperature of more than 140° C. because although it is possible to decrease various pheophorbide amounts or carry out sterilization in a very short time, the content of β-carotene in Dunaliella powder is also lowered due to oxidative degradation. Furthermore, time necessary for the heat treatment step is preferably in the range from 2 to 80 min, and more preferably in the range from 5 to 60 min. It is not preferable that heat treatment time is less than 2 min because various pheophorbide amounts cannot be decreased or sterilization sufficiently for selling as health foods and the like cannot be performed. It is not preferable that heat treatment time is longer than 80 min because β-carotene cannot be obtained at a high content due to oxidative degradation. Note here that the heat treatment step is not necessarily carried out after the desalting treatment step, but it may be carried out in arbitrary orders, for example, it is carried out before the pH adjusting treatment step.

Then, paste, which has been obtained after a neutralization treatment step or heat treatment step if necessary, is formed into a dried powder product by removing water from the paste by well-known methods such as spray drying, or lyophilization under decreased pressure.

The Dunaliella powder obtained in the above-mentioned series of methods has a total pheophorbide amount of 160 mg % or less and an existing pheophorbide amount of 100 mg % or less, and contains 3 to 20 g of β-carotene in 100 g of the Dunaliella powder. Furthermore, the amount of β-carotene contained in 100 g of the Dunaliella powder differs depending upon the algae belonging to the genus Dunaliella to be used as a raw material, but the amount is more preferably 5 to 15 g, and most preferably 6 to 10 g. It is not preferable that the amount of β-carotene contained in 100 g of the Dunaliella powder is less than 3 g because a commercial value thereof is lowered. Furthermore, in order to achieve a content of 20 g or higher, the algae belonging to the genus Dunaliella as the raw material is required to contain more β-carotene.

In yet some further embodiments, the Dunaliella preparation of the present disclosure is based on Dunaliella bardawil prepared as detailed below. It should be noted that the family Dunaliellaceae, and specifically, the genus Dunaliella is a single-celled, photosynthetic green alga, that is characteristic for its ability to outcompete other organisms and thrive in hypersaline environments. Certain species of this genus can accumulate relatively large amounts of β-carotenoids and glycerol in very harsh growth conditions consisting of high light intensities, high salt concentrations, and limited oxygen and nitrogen levels. Dunaliella bardawil is well-known microalgae accumulating high levels of beta-carotene under growth-limiting conditions, that is primarily composed of the isomers 9-cis and all-trans.

In some embodiments, the Dunaliella used by the invention is grown in any growth conditions, for example, any salinity conditions, as well as any light conditions and any temperature conditions. Non-limiting examples for salinity conditions include salt concentrations of 1M, 2M, 3M or more NaCl, up to 4M.

It should be understood that the Dunaliella preparations used in the present invention are prepared from any Dunaliella species, strains and isolates. In some specific and non-limiting embodiments, the Dunaliella preparations as used herein are prepared from Dunaliella Bardawil. In yet some more specific embodiments, Dunaliella bardawil as used herein is the Ben-Amotz and Avron, isolated from salt pond near Bardawil Lagoon, North Sinai, 1976. In yet some further specific embodiments, the Dunaliella bardawil is as used herein as denoted by ATCC® 30861™. It should be understood that the invention further encompasses the use of any progeny, strain, isolate, mutant or variant of the Dunaliella bardawil as denoted by ATCC® 30861, for any of the aspects described by the invention.

Dunaliella bardawil (hereinafter “db”) was grown and cultivated in large body open salt water ponds of 50,000 m² to obtain algae comprising approximately 8% by weight of β-carotene (hereinafter “BC”) at an approximately 1:1 (by weight) ratio of 9-cis and all-trans isomers of BC, or greater than 1:1 ratio of 9-cis and all-trans isomers of BC. The algae were harvested by dislodging centrifuges into a concentrated paste. The paste was washed to remove salt and sterilized, and then spray dried to yield db powder comprising approximately 8% BC and less than 5% moisture. The powder was packaged in capsules of 250-300 mg algae containing 15-20 mg of BC each together with all of the natural components of the algae. The BC of the capsules retains the original ratio of isomers. The capsules were packaged in vacuum closed blisters which have a shelf life of up to three years.

In specific embodiments the Dunaliella preparation of the present disclosure is encapsulated. Encapsulation is the process used to entrap one substance (termed core material or active agent) within another (coating, shell, or carrier/wall material). More specifically, a dried powder of Dunaliella algae, a tablet obtained by compressing and hardening the dried powder of Dunaliella algae, or a capsule obtained by encapsulating the dried powder of Dunaliella algae are known. In any states, firstly, it is necessary to dry a culture solution of Dunaliella algae and to form it into dried powder.

For example, Japanese Patent Laid-Open No. 1997-203 discloses that a dried powder product of the algae belonging to the genus Dunaliella is obtained by previously decreasing the water content of a culture solution of cultured Dunaliella alga body to, preferably, about 50% for easy drying, followed by being subjected to nebulization drying, vacuum drying or freeze drying.

Dried powder products of the algae belonging to the genus Dunaliella are sold as foods. It is necessary to carry out a step of decreasing compounds that may be harmful to a human body in a step of producing a dried powder product from the harvested algae belonging to the genus Dunaliella in order to satisfy a predetermined safety standard.

As detailed above, in various embodiments the Dunaliella algae preparation of the present disclosure may be a Dunaliella bardawil preparation.

In specific embodiments the Dunaliella algae preparation of the present disclosure may be adapted for add-on to a beverage, solid, semi-solid or liquid food, food additive, food supplement, medical food, botanical drug, drug and/or a pharmaceutical compound.

In still further particular embodiments the Dunaliella algae preparation of the present disclosure is used as a functional food. By the term “functional foods” it is meant whole, fortified, enriched or enhanced foods that provide health benefits beyond the provision of essential nutrients (e.g., vitamins and minerals), when they are consumed at efficacious levels as part of a varied diet on a regular basis.

In further particular embodiments the Dunaliella preparation of the present disclosure is used as a food supplement. A food supplement, the term coined by the European Commission for Food and Feed Safety, or a dietary supplement, an analogous term adopted by the US Food and Drug Administration (FDA), relates to any kind of substances, natural or synthetic, with a nutritional or physiological effect whose purpose is to supplement the normal diet. In this sense, this term also encompasses food additives and dietary ingredients. Further, under the Dietary Supplement Health and Education Act of 1994 (DSHEA), a statute of US Federal legislation, the term dietary supplement is defined as a product (other than tobacco) intended to supplement the diet that bears or contains one or more of the following dietary ingredients: a vitamin, a mineral, an herb or other botanical, an amino acid, a dietary substance for use by man to supplement the diet by increasing the total dietary intake, or a concentrate, metabolite, constituent, extract, or combination of any of the aforementioned ingredients.

Under food or dietary supplements is meant those marketed in a form of pills, capsules, powders, drinks, and energy bars and other dose forms. The European and the US laws regulate dietary supplements under a different set of regulations than those covering “conventional” foods and drug products. According thereto, a dietary supplement must be labeled as such and be intended for ingestion and must not be represented for use as conventional food or as a sole item of a meal or a diet.

In yet some further embodiments, the Dunaliella algae preparation of the present disclosure may be used as an add-on to medical foods. By “medical foods” it is meant foods that are specially formulated and intended for the dietary management of a disease that has distinctive nutritional needs that cannot be met by normal diet alone. The term medical food, as defined in the FDA's 1988 Orphan Drug Act Amendments is a food which is formulated to be consumed or administered entirely under the supervision of a physician and which is intended for the specific dietary management of a disease or condition for which distinctive nutritional requirements, based on recognized scientific principles, are established by medical evaluation.

Also pertinent to the present context are botanical drugs. In specific embodiments, the Dunaliella algae preparation of the present disclosure may be an add-on to a botanical drug. As used herein the term “botanical drug” refer to products that are intended for use in the diagnosis, cure, mitigation, treatment or prevention of disease in humans. A botanical drug product consists of vegetable materials, which may include plant materials, algae, macroscopic fungi, or combinations thereof. A botanical drug product may be available as (but not limited to) a solution (e.g., tea), powder, tablet, capsule, elixir, topical, or injection. Botanical drug products often have unique features, for example, complex mixtures, lack of a distinct active ingredient, and substantial prior human use. Fermentation products and highly purified or chemically modified botanical substances are not considered botanical drug products. According to the FDA Guidance for Industry, a botanical product may be a food (including a dietary supplement), a drug (including a biological drug), a medical device (e.g., gutta-percha), or a cosmetic. Further, botanical drugs may include botanical ingredients in combination with either a synthetic or highly purified drug or a biotechnology derived or other naturally derived drug. In the same way, botanical drugs may also contain animals or animal parts (e.g., insects, annelids, shark cartilage) and/or minerals or a combination thereof.

As indicated above, the method according to the present disclosure comprises administering to the subject an effective amount of at least one Dunaliella algae preparation or any composition comprising thereof.

The term “effective amount” means an amount necessary to achieve a selected result. The effective amount is determined by the severity and type of the disease or condition in conjunction with the preventive or therapeutic objectives, the route of administration and the patient's general condition (age, sex, weight and other considerations known to the attending physician). The effective amount may be determined based on animal models, such as these presented in the Examples.

In some embodiments the Dunaliella algae preparation of the present disclosure is comprised in a composition.

The composition comprising the Dunaliella algae preparation of the present disclosure may be prepared according to any method known in the art.

In particular embodiments the composition comprising the Dunaliella algae preparation of the present disclosure is a pharmaceutical composition.

The pharmaceutical compositions comprising the Dunaliella algae preparation of the present disclosure generally comprise a buffering agent, an agent which adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

It should be therefore appreciated that the pharmaceutical compositions of the invention as well as all Dunaliella algal preparations described above may be applicable for any of the neurodegenerative disorders discussed above, specifically, any conditions associated with aggregation of beta-amyloid, any of the tauopathies mentioned above and/or any early signs or symptoms associated therewith.

Administering the Dunaliella algae preparation of the present disclosure or any composition comprising the same may be performed by any route known in the art, enteral or parenteral. In some embodiment the method according to the present disclosure is wherein said Dunaliella algae preparation is administered orally.

According to certain embodiments, the Dunaliella algal preparations of the invention or any composition thereof may be administered by oral, intravenous, intramuscular, subcutaneous, intraperitoneal, parenteral, transdermal, intravaginal, intranasal, mucosal, sublingual, topical, rectal or subcutaneous administration, or any combination thereof.

According to a specific embodiment, the composition of the invention may be particularly suitable for oral or mucosal administration use. The usefulness of an oral formulation requires that the active agent or preparations of the invention be bio-available. Bioavailability of orally administered drugs can be affected by a number of factors, such as drug absorption throughout the gastrointestinal tract, stability of the drug in the gastrointestinal tract, and the first pass effect. Thus, effective oral delivery of an active agent or combination requires that the active agent have sufficient stability in the stomach and intestinal lumen to pass through the intestinal wall. Many drugs, however, tend to degrade quickly in the intestinal tract or have poor absorption in the intestinal tract so that oral administration is not an effective method for administering the drug.

More specifically, the Dunaliella algal preparations and composition of the invention may be suitable for mucosal administration, for example, pulmonary, buccal, nasal, intranasal, sublingual, rectal, vaginal administration and any combination thereof.

Pharmaceutical compositions suitable for oral administration are typically solid dosage forms (e.g., tablets) or liquid preparations (e.g., solutions, suspensions, or elixirs).

Solid dosage forms are desirable for ease of determining and administering dosage of active ingredient, and ease of administration, particularly administration by the subject at home.

Liquid dosage forms also allow subjects to easily take the required dose of active ingredient. Liquid preparations can be prepared as a drink, or to be administered, for example, by a nasal-gastric tube (NG tube). Liquid oral pharmaceutical compositions generally require a suitable solvent or carrier system in which to dissolve or disperse the active agent, thus enabling the composition to be administered to a subject. A suitable solvent system is compatible with the active agent and non-toxic to the subject. Typically, liquid oral formulations use a water-based or an oil-based solvent.

The oral compositions of the invention can also optionally be formulated to reduce or avoid the degradation, decomposition, or deactivation of the active agents by the gastrointestinal system, e.g., by gastric fluid in the stomach. For example, the compositions can optionally be formulated to pass through the stomach unaltered and to dissolve in the intestines, i.e., enteric coated compositions.

Oral compositions can also be prepared using an excipient. Pharmaceutically compatible binding agents, and/or adjuvant materials can be included as part of the composition. Oral dosage forms comprising Dunaliella algal preparations are provided, wherein the dosage forms, upon oral administration, provide a therapeutically effective blood level of Dunaliella algal preparations to a subject. Also provided are dosage forms comprising said Dunaliella algal preparations wherein the dosage forms, upon administration, provide a therapeutically effective blood level of the Dunaliella algal preparations to a subject. For the purpose of mucosal therapeutic administration, the active combined compounds (e.g., Dunaliella algal preparations) can be incorporated with excipients or carriers suitable for administration by inhalation or absorption, e.g., via nasal sprays or drops, or rectal or vaginal suppositories.

In some further specific embodiment the method according to the present disclosure is wherein said Dunaliella algae preparation is administered in combination with at least one additional agent. In other words, the methods of the present disclosure encompass combination therapy with at least one additional therapeutic agent, the type of additional therapeutic agent depending on the type of the disease or condition being treated.

The term “combination therapy” can mean concurrent or consecutive administration of two or more agents. For example, concurrent administration can mean one dosage form in which the two or more agents are contained whereas consecutive administration can mean separate dosage forms administered to the subject at different times and optionally by different routes of administration.

In another aspect thereof, the present disclosure further provides at least one Dunaliella algae preparation or any composition comprising thereof for use in a method for preventing, treating, ameliorating, reducing or delaying the onset of at least one of a neurodegenerative disease, a disorder associated with protein misfolding, cognitive decline and any conditions or symptoms associated therewith in a subject in need thereof.

In some embodiments, the at least one Dunaliella algae preparation or any composition comprising thereof for use according to the present disclosure is wherein said method results in amelioration or reduction of at least one symptom associated with at least one of a neurodegenerative disease, a disorder associated with protein misfolding, and cognitive decline in a subject in need thereof.

In other embodiments, the at least one Dunaliella algae preparation or any composition comprising thereof for use according to the present disclosure is wherein said symptom is at least one of short term memory impairment, long term memory impairment, impaired cognitive function, impaired learning function, β-amyloids deposition, anxiety, depression or any combination thereof.

In further embodiments, the at least one Dunaliella algae preparation or any composition comprising thereof for use according to the present disclosure is wherein said method results in improvement of at least one of cognitive function, short term memory, long term memory, acquisition time and clearance of β-amyloids in a subject in need thereof.

In still further embodiments, the at least one Dunaliella algae preparation or any composition comprising thereof for use according to the present disclosure is wherein said neurodegenerative disease is at least one of Alzheimer's disease, Parkinson's disease, Mild Cognitive Impairment (MCI), Parkinson's disease with MCI, Huntington's disease, Lewy body disease, Amyotrophic lateral sclerosis (ALS), Prion disease, Motor neuron disease (MND), Spinocerebellar ataxia (SCA), Spinal muscular atrophy (SMA), Friedreich's Ataxia and any other neurodegenerative-related dementia or ataxia.

In still further particular embodiments, the at least one Dunaliella algae preparation or any composition comprising thereof for use according to the present disclosure is wherein said neurodegenerative disease is Alzheimer's disease.

In various specific embodiments the at least one Dunaliella algae preparation or any composition comprising thereof for use according to the present disclosure is for preventing, treating, ameliorating, reducing or delaying the onset of cognitive decline, Specifically, as defined in connection with other aspects of the invention.

In various specific embodiments the at least one Dunaliella algae preparation or any composition comprising thereof for use according to the present disclosure is wherein said method further comprises administration of at least one additional agent.

Still further, in some embodiments the at least one Dunaliella algae preparation or any composition comprising thereof for use according to the present disclosure is wherein said Dunaliella algae is Dunaliella bardawil.

In particular embodiments, the at least one Dunaliella algae preparation or any composition comprising thereof for use according to the present disclosure is wherein said Dunaliella algae preparation is administered orally.

In yet another aspect, the present disclosure provides the use of at least one Dunaliella algae preparation for the manufacture of a composition for preventing, treating, ameliorating, reducing or delaying the onset of at least one of a neurodegenerative disease, a disorder associated with protein misfolding, cognitive decline and any conditions or symptoms associated therewith in a subject in need thereof.

In some embodiments the use according to the present disclosure is wherein said composition ameliorates or reduces at least one symptom associated with at least one of a neurodegenerative disease, a disorder associated with protein misfolding, and cognitive decline in a subject in need thereof.

In other embodiments the use according to the present disclosure is wherein said symptom is at least one of short term memory impairment, long term memory impairment, impaired cognitive function, impaired learning function, s-amyloids deposition, anxiety, depression or any combination thereof.

In further embodiments the use according to the present disclosure is wherein said composition improves at least one of cognitive function, short term memory, long term memory, acquisition time and clearance of β-amyloids in a subject in need thereof.

In still further particular embodiments the use according to the present disclosure is wherein said neurodegenerative disease is at least one of Alzheimer's disease, Parkinson's disease, Mild Cognitive Impairment (MCI), Parkinson's disease with MCI, Huntington's disease, Lewy body disease, Amyotrophic lateral sclerosis (ALS), Prion disease, Motor neuron disease (MND), Spinocerebellar ataxia (SCA), Spinal muscular atrophy (SMA), Friedreich's Ataxia and any other neurodegenerative-related dementia or ataxia.

In various particular embodiments the use according to the present disclosure is wherein said neurodegenerative disease is Alzheimer's disease.

In still further embodiments the use according to the present disclosure is for preventing, treating, ameliorating, reducing or delaying the onset of cognitive decline, specifically, as described herein in connection with other aspects of the invention.

In various embodiments the use according to the present disclosure is wherein said Dunaliella algae preparation is administered in combination with at least one additional agent.

In additional embodiments the use according to the present disclosure is wherein said Dunaliella algae is Dunaliella bardawil.

In other embodiments the use according to the present disclosure is wherein said Dunaliella algae preparation is administered orally.

In yet another aspect, the invention provides at least one Dunaliella algae preparation or any composition comprising thereof for use in a method for improving at least one of short term memory impairment, long term memory impairment, impaired cognitive function, impaired learning function, β-amyloids deposition in a subject in need thereof. In yet some further aspects, the invention provides a method for improving at least one of short term memory impairment, long term memory impairment, impaired cognitive function, impaired learning function, β-amyloids deposition in a subject in need thereof. In some embodiments, the method of the invention comprises the step of administering to the subject an effective amount of at least one Dunaliella algae preparation or any composition comprising thereof.

In further embodiments the method according to the present disclosure results in improvement of at least one of cognitive function, short term memory, long term memory, acquisition time and clearance of β-amyloids in a subject in need thereof.

In other embodiments the method according to the present disclosure is for preventing, treating, ameliorating, reducing or delaying the onset of cognitive decline.

In particular embodiments the method according to the present disclosure is wherein the Dunaliella algae is Dunaliella bardawil.

In still further specific embodiments, the method according to the present disclosure is wherein the Dunaliella algae preparation is administered orally.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

All scientific and technical terms used herein have meanings commonly used in the art unless otherwise specified. The definitions provided herein are to facilitate understanding of certain terms used frequently herein and are not meant to limit the scope of the present disclosure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. As used herein the term “about” refers to ±10%.

The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to specifically disclose sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and arm meant to include the first and second indicated numbers and all the fractional and integral numerals there between.

Various embodiments and aspects of the present invention as delineated hereinabove and as claimed in the claims section below find experimental support in the following examples.

The following examples are representative of techniques employed by the Inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It must be noted that, as used in this specification and the appended claims, the singular forms “a”, “an” and “the” include plural referents unless the content clearly dictates otherwise.

### EXAMPLES

Reference is now made to the following examples, which together with the above descriptions illustrate some embodiments of the invention in a non-limiting fashion.

### Experimental Procedures

### Animals

The following mice models were used:
Tg2576 mice (Taconic Biosciences, Inc.) [27] and age-, sex-, and strain-matched C57Bl6 wild type (WT) mice. Tg2576 mice were self-bread in the Inventors' animal facility. Tg2576 mice express the human 695-aa isoform of the amyloid precursor protein (APP) containing the Swedish double mutation (APPswe) driven by a hamster prion promoter. The Tg2576 model has been chosen since it is well-characterized. Furthermore, in this model, mice pathology develops relatively slowly, but rapidly enough for obtaining substantive findings, and thus this model is more pertinent to future human applications. Additionally, these mice have a relatively high survival rate (it has been previously shown that by 12 months approximately 75% of the mice survive). Since Tg2576 mice tend to be aggressive, animals were housed one animal per cage at the SPF (specific pathogen free) approved Sheba Animal Facility.
5×FAD mice. These mice were purchased from Jackson Laboratory (Bar Harbor, Me., USA). 5×FAD mice overexpress mutant human amyloid precursor protein (APP) with the Swedish (K670N, M671L), Florida (I716V), and London (V717I) mutations along with mutant human presenilin 1 (PS1) with two FAD mutations (M146 and L286V).

Tg2576 mice were treated for 10 months (from 2 to 12 months of age). Weight was obtain every 2 months. Two weeks before sacrifice, behavioral tests were performed (including open field, measuring general locomotors activity, anxiety and willingness to explore, Y-maze spontaneous alternation, measuring exploratory and spatial working memory and Barnes maze, measuring long term memory). Animals were sacrificed at the age of 12 months.

5×FAD mice were treated for 6 months (from approximately 1 to 6 months of age). Weight was monitored monthly. Two weeks before sacrifice, behavioral tests were performed (including open field, measuring general locomotors activity, anxiety and willingness to explore, Y-maze and Novel object recognition, measuring exploratory and spatial working memory Novel object recognition and Barnes maze, measuring long term memory). Animals were sacrificed at the age of 7 months.

### Dietary Conditions

When animals reached eight weeks of age, Tg2576 and WT control mice were randomly allocated into two groups each (12 mice in each group) and were fed for 10 months on 8% Dunaliella algae powder diet (also referred to herein as the “Dunaliella diet”) or on control diet. Low-fat chow diet (18% protein, 5% fat; TD2018, Harlan Teklad) was used as basic (control) diet. To prepare the food, 750 ml distilled hot water were mixed with 28 gram gelatin until the solution was clear. Then, 1 kilogram powder of the low-fat chow diet (control) or low-fat chow diet with Dunaliella algae powder (80 g/kg feed) were well mixed with the warm gelatin solution. After solidifying, the food was divided into tablets and stored in −80° C. (thereby, except for the Dunaliella algae powder content, the two diets had essentially the same content and texture). Feed was replaced every two days to minimize oxidation and degradation of the ingredients.

### Dunaliella Algae Powder Preparation

Dunaliella bardawil (hereinafter “db”, Nikken Sohonsha Corporation) was grown and cultivated in large body open salt water ponds of 50,000 m² to obtain algae comprising approximately 5-8% by weight of β-carotene (hereinafter “BC”) at an approximately 1:1 (by weight) ratio of 9-cis and all-trans isomers of BC, or greater than 1:1 ratio of 9-cis and all-trans isomers of BC. The algae were harvested by dislodging centrifuges into a concentrated paste. The paste was washed to remove the salt and sterilized, and then spray dried to yield Dunaliella bardawil powder comprising approximately 5-8% BC and less than 5% moisture. The powder was packaged in capsules of 250-500 mg algae containing BC (5-8%) together with all of the other natural components of the algae (e.g. protein, lipids, carbohydrates). The BC of the capsules retains the original ratio of isomers. The capsules were packaged in vacuum closed blisters which have a shelf life of up to three years.

The Dunaliella algae powder preparation used herein contains about 7% β-carotene composed of 40%-50% 9-cis β carotene (9βC) and 50%-60% all trans β carotene.

### Blood Brain Barrier Models

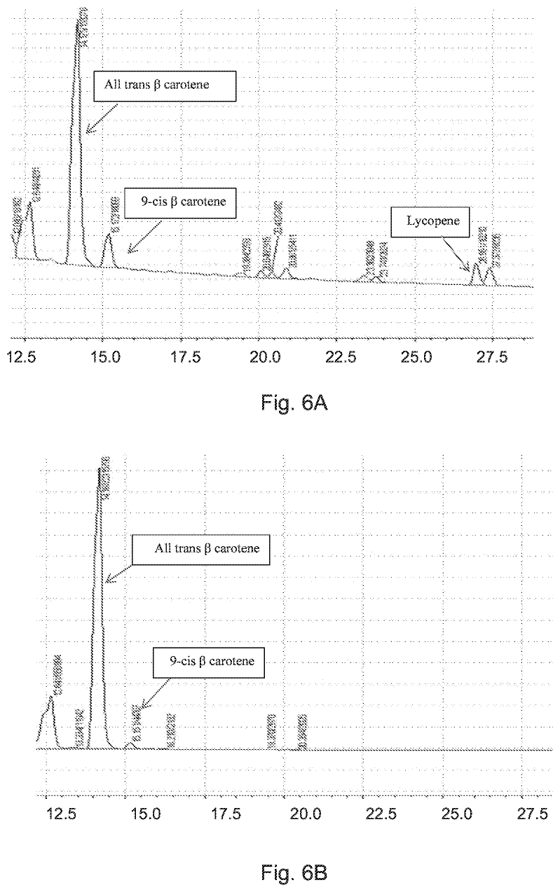

An in vitro model of the blood brain barrier (BBB) was used [28]. The model consists of a monolayer of endothelial cells, obtained from primary cultures of endothelial cells established from freshly collected porcine brain forming tight junctions that are grown on a microporous membrane filter culture insert (the “Luminal side” in FIG. 6) and of a monolayer of glial cells extracted from new born rats' cortex that are seeded at the abluminal side of the filter (the “Abluminal side” in FIG. 6). Low density lipoprotein (LDL, 100 μl, 1,600 μg/ml from a healthy volunteer) was added to the luminal side. Cells were then incubated for 24 hours and thereafter samples were collected and analyzed using HPLC. The model was prepared by first seeding the glial cells on the abluminal side of a filter, a week later, the above-described endothelial cells were seeded on the luminal side of the filter and two days later LDL was added to the luminal side of the filter as indicated above.

In order to examine whether the 9βC and all trans β-carotene (βC) isomers of the Dunaliella algae powder preparation of the invention crossed the BBB, the β-carotene isomer levels, and the levels of other carotenoids and their metabolites were also determined in vivo, in the above described Tg2576 and 5×FAD mice model, in the plasma, liver and in the different brain regions (hippocampus and frontal cortex) by high performance liquid chromatography (HPLC).

It is also examined whether retinol and retinoic acid cross the BBB in order to discover which can be a source for brain's retinoids. In addition, in order to examine whether β-carotene can be converted into retinol in the brain different cells are isolated from the brain (hippocampal neurons and astrocytes) and the expression (mRNA and protein) of β-carotene 15,15′-monooxygenase 1 (BCMO1) and its activity (retinol formation) in the cells are measured.

### LDL Isolation for the BBB In Vitro Model

LDL was obtained from healthy volunteers by sequential ultracentrifugation (density, 1.063 g/ml), and the concentration was determined by the Lowry method.

### Behavioral Tests—Learning and Memory Tests

As detailed above, 30 eight week old Tg2576 mice and 30 C57Bl/6 WT mice were randomly allocated into two groups each and placed for 10 months on two different diets: regular chow diet (control) and Dunaliella diet (8% of 9-cis β-carotene rich Dunaliella algae powder in 1 Kg feed). The following behavioral tests were performed: open field, Y-maze and Barnes maze.

### Barnes Maze

The Barnes maze is a spatial-learning task that allows animals to use spatial cues to locate a means of escape from a mildly aversive environment [29]. Both the Barnes maze and the Morris water maze (MWM) examine spatial memory, but unlike the MWM, the Barnes maze lack the stress induced by swimming. During the task, mice are placed in a cylindrical dark chamber at the center of a circular table containing 18 holes around the edge. After 10 seconds the chamber is lifted and the animal receive negative reinforcements, such as bright light, loud buzzer, an exposed environment, and air jets [30, 31], in order to motivate them to find the escape hole leading to a drawer underneath one of the holes. The animal explores the maze until it finds and enters the escape box in 180 seconds. If the mouse fails to enter the escape box within 180 seconds it is picked up gently by the base of the tail, placed in the palm of the hand and let down at the side of the escape hole. The mouse enters the escape box and remains there for an additional 60 seconds before it is removed and taken to its home cage.

After each trial, the maze and escape box are cleaned thoroughly with a 10% alcohol solution to remove odors. During the 180 seconds of escape latency training, the number of errors and path length are measured. The escape latency is the duration of time between removal of the cylinder and the animal's entry into the escape box. The animals will be subjected to 4 trials per day for 4 days. On the fifth day a recall test will be performed. The escape box will be removed and the same parameters will be measured. A second recall test will be performed on day 12 to assess long term retention, of where the target escape box was located.

### Y Maze

The Y maze spontaneous alternation is a behavioral test for measuring the willingness of rodents to explore new environments. Y-maze test allows assessment of spatial working memory that is dependent upon the hippocampus [32]. The mouse at the age of 12 months is placed at the end of one arm of the Y-maze and allowed to freely explore the maze for 6 minutes. Alternation will be determined from successive entries into the three arms on overlapping triplet sets in which three different arms are entered. An actual alternation will be defined as entries into all three arms consecutively (i.e., ABC, CAB, or BCA but not BAB). An entry will be defined as placing all four paws within the boundaries of the arm. The maze arms will be cleaned with 30% ethanol between tasks to remove residual odors.

### Open Field Test

The open field test is a commonly used qualitative and quantitative measure of general locomotor activity, anxiety and willingness to explore in rodents. The mouse is placed at the corner of the test box and allowed to freely explore the area for 5 minutes. Four measurements are recorded: (a). total path, which indicates general activity and exploratory behavior; (b). percentage of cell used, which indicates general activity and exploratory behavior; (c). percentage of time moving, which indicates anxiety and general activity; and (d). sum center, which is the sum of the time the mouse spends in the arena's center that indicates anxiety. The test is recorded and analyzed. Higher scores in each of these measurements reflect lower anxiety and higher locomotor activity.

### Novel Object Recognition

The mouse is presented with two identical objects for 5 minutes, 3 hours later for short term memory, 24 hours for long term memory; one of the objects is replaced by a different one. The amount of time taken to explore the new object provides an index of recognition memory.

### Measurement of Hippocampal Formation of Aβ Peptides

Measurements were performed on brain extracts of mice (Tg2576 and 5×FAD) after they were sacrificed. Brain tissues were homogenized and samples of proteins extracted therefrom were analyzed. Amyloid beta levels were measured using the Human β amyloid (1-42) ELISA kit (Wako). Briefly, for quantitative assessment of hippocampal formation Aβ peptides, frozen pulverized tissue was extracted in a two-step extraction. The tissue was homogenized in 1% Triton X 100, diluted in 25 mm phosphate buffered saline containing 137 mm NaCl and protease inhibitors cocktail (Roche Biochemicals, Indianapolis, Ind., USA) and centrifuged for 1 hour at 4° C. at 100,000 g. The soluble fraction was designated “Aβsol”. The remaining pellet was then sonicated in 5 m guanidine HCl with 50 mm Tris PH=8 and protease inhibitors cocktail, incubated for 2 hours at 25° C., and centrifuged at 13 000 g for 20 minutes at 4° C. The latter (insoluble) fraction was designated “Aβinsol”. Aβ1 42 was measured by sandwich ELISA (WAKO, Osaka, Japan) according to the manufacturer's instructions.

Expression of RXR and Downstream Genes in Animals Fed on Dunaliella Algae Powder Preparation Diet—NanoString nCounter Gene Expression Analysis

Using the Nanostring method it was examined whether 9-cis βC (present in the Dunaliella algae powder preparation diet) affects genes associated with inflammation, BBB synaptic plasticity and lipidation via retinoic X receptor (RXR) in the brain and in other tissues. For this matter, total RNA was extracted from mice hippocampus from one hemisphere, using NucleoSpin® RNA (Macherey-Nagel) or by PARIS™ Kit (Invitrogene™), designed for RNA and protein extraction from the same tissue. Extracted RNA was subjected to NanoString gene expression analysis. The standard NanoString protocol was followed. RNA (70 ng) was used to assess the expression of 43 mouse genes that were possibly Alzheimer's' (AD) related and RXR affected, divided to different pathways: Inflammation, lipidation, BBB and synaptic plasticity. Inclusion criteria: Values with Ratio higher than 1.4 or lower than 0.7 and p<0.05 were considered significant and used for statistical analysis.

### Western Blot Analysis

Mice hippocampus were lysed using PARIS™ Kit (Invitrogen™) or RIPA buffer. The protein lysis was denatured for 5 minutes at 95° C. in SDS-PAGE sample buffer and separated by 12% SDS-PAGE. Proteins were transferred onto nitrocellulose membranes. Membranes were incubated with blocking buffer for 1 hour prior to incubation with primary antibodies as required: anti-GFAP (Abcam ab53554, 1:500), anti-Synaptophysin (abcam [YE269] ab 32127, 1:10000), PBR (Santa Cruz, FL-169:sc-20120, 1:100), a Tubulin (Santa Cruze, B-7:sc-5286, 1:100), GAPDH (1:150). Proteins were visualized using IRDye® 680CW Goat anti-Rabbit (1:15000) and IRDye® 680CW Donkey anti-Mouse (1:15000), IRDye®800CW Donkey anti-Goat (1:15000), IRDye® 800CW Goat anti-Mouse (1:15000) secondary antibodies. Images were captured with the Odyssey system (CLX). The proteins were quantified using Image Studio Ver 5.2 software.

### Carotenoids Analysis

Mice Brain tissue were homogenized with 2 mL ethanol containing 10 μM butylated hydroxytoluene which was followed by the addition of 2 mL hexane and 1 mL of Double-distilled water (DDW). The samples were mixed and centrifuged for 5 minutes at 1000×g. The hexane layer was separated and dried under a stream of N₂. Dried samples were suspended in 100 μL methyl-tert-butyl-ether, and e concentrations were determined by reverse phase HPLC on a YMC C30 column (CT995031546QT, 150×4.6, 3 μm particle size; YMC Inc., Allentown, Pa., USA) with methanol/methyl-tert-butyl-ether/water 1.5% ammonium acetate as the mobile phase at a flow rate of 1 mL/min. βc was detected by monitoring its absorbance at 450 nm and by comparison with the retention times of authentic standards.

### Cholesterol and TG Measurement

Colorimetric enzymatic procedures were used to measure plasma total cholesterol (Chol, Roche/Hitachi, Roche Diagnostics) and triglycerides (Infinity, Thermo Electron Corporation). Cobas Mira autoanalyzer (Roche) or Beckman coulter AU-480 was used for lipid measurements.

### Statistical Analysis

Data were reported as mean±standard error of the mean. Results were analyzed by student t-test. Statistical significance was obtained when p values were less than 0.05.

### Example 1

### Survival and Weight Gain of Mice Fed on Dunaliella Algae Powder Preparation

As detailed above, thirty Tg2576 mice and thirty C57Bl/6 WT mice were randomly allocated at the age of two months into two groups each and placed for 10 months on two different diet types: regular chow diet and Dunaliella algae powder preparation diet (80 gr of 9-cis β-carotene rich Dunaliella algae powder in 1 Kg chow diet feed) in a 2 (mice type) by 2 (diet type) design.

As previously shown, Tg2576 mice had a lower survival rate in comparison to WT mice. However, as shown in Table 1 below, Tg2576 mice that were fed with the Dunaliella algae powder preparation diet, had substantially higher survival rates compared to Tg2576 mice fed with regular diet (p=0.052).

TABLE 1
Survival percentage of the different groups

| | Survival |
|---|---|
| Group | percentage |
| WT Dunaliella algae preparation | 91.6% |
| WT control | 91.6% |
| Tg2576 Dunaliella algae preparation | 78.5% |
| Tg2576 control | 45.0% |

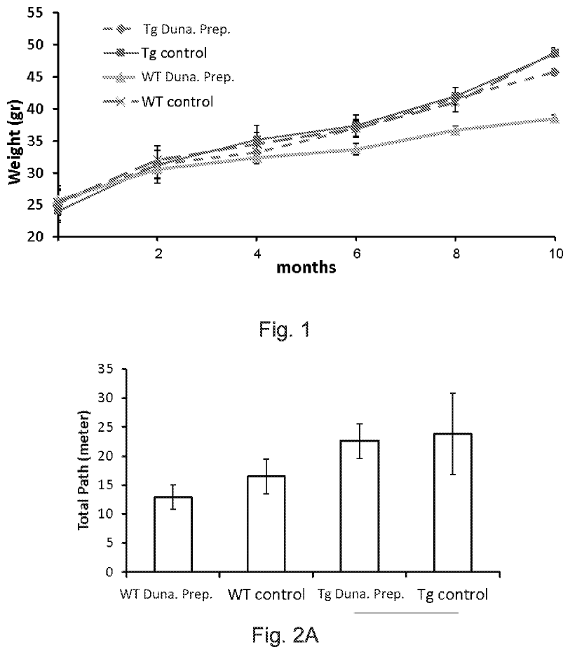

During the experiment, the body weight of the animals was measured every two months. As shown in FIG. 1, at baseline the animals had similar weight, however in the following measurements a growing difference between wild type mice fed on Dunaliella algae powder preparation diet (WT Duna. prep.) and the other groups was detected. At the end of the experiment, WT mice fed on Dunaliella algae powder preparation diet gained an average of 12 gr, while the other groups gained an average of 22 gr. A significant difference (p<0.05) is shown between WT mice fed on Dunaliella algae powder preparation diet compared to WT control mice (wild type mice fed on regular diet).

It is known in the art that adipose tissues are important site of carotenoids and retinol storage and there are several studies showing that retinoids have an anti-adiposity and anti-inflammatory action in obesity. Therefore, the effect of Dunaliella diet on the mouse weight in the present experiment is reasonable. The reason for the different effect in WT mice fed on Dunaliella algae powder preparation diet as compared to WT mice fed on the control diet is unknown. Nevertheless, reduction of body weight by Dunaliella algae powder preparation treatment has not been demonstrated before.

Interestingly, as shown in FIG. 1, there was no significant difference in weight gain between Tg Duna. Prep. mice and the WT control mice, namely between Tg2576 mice fed on the Dunaliella algae powder preparation diet and wild type control mice. In addition, apparently the Dunaliella algae powder preparation diet ameliorated the weight gain observed in the Tg2576 mice on regular diet.

### Example 2

### Locomotor Activity, Anxiety and Exploratory Behavior in Mice Fed on Dunaliella Algae Powder Preparation

In the present and following Examples, the effect of the Dunaliella algae powder preparation diet on cognitive functions was examined in the Alzheimer's disease (AD) mouse model using behavioral tests. First, the open field test was used in order to investigate the general locomotors activity, anxiety and exploratory behavior in WT and Tg2576 mice fed with Dunaliella algae powder preparation diet as compared to control mice.

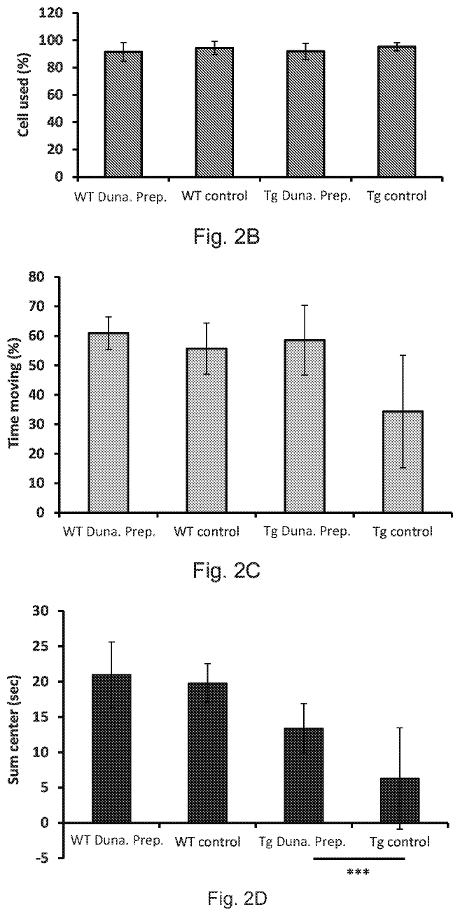

As demonstrated in FIG. 2A and FIG. 2B, there were no significant effects for the Dunaliella algae powder preparation diet on the “total path” and “percentage of cells used” properties in this behavioral model. However, as shown in FIG. 2C and FIG. 2D, there was a significant effect on the “percentage of time moving” (FIG. 2C) and the “sum center” (FIG. 2D). Tg2576 mice had longer path but spent less time in the center of the arena than WT mice, which indicates higher anxiety. As demonstrated in FIG. 2C, in Tg2576 mice fed on Dunaliella algae powder preparation diet (Tg Duna. Prep.), the percentage of time moving, which indicates anxiety and general activity, was approximately the same as for the WT mice. Furthermore, although the time spent in the arena's center of Tg Duna. Prep. mice was lower than for the WT mice as demonstrated in FIG. 2D, this score was higher than that obtained in the Tg control mice

### Example 3

### Willingness to Explore New Environments of Mice Fed on Dunaliella Algae Powder Preparation

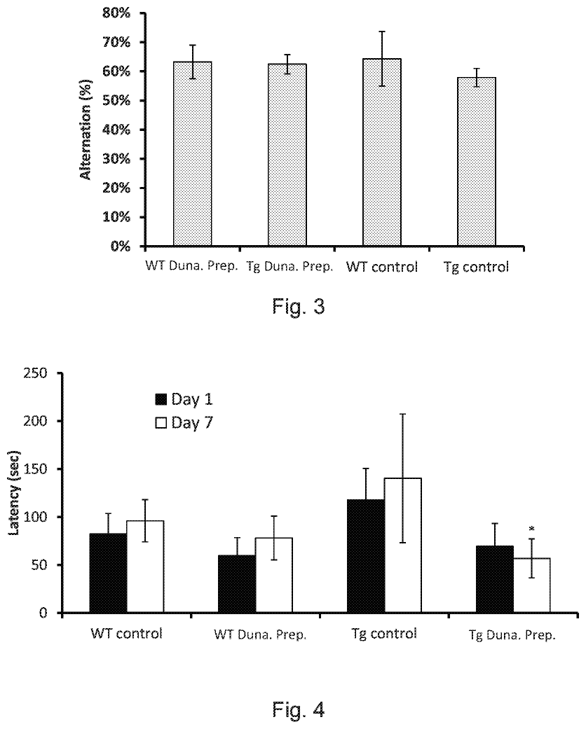

In this example the Y-maze test was used. The alternation's percentage defined as entries into all three arms of the Y-maze consecutively was calculated in order to measure the willingness of the mice (Tg2576) to explore new environments and to assess spatial working memory. As evident from FIG. 3, Dunaliella algae powder preparation diet did not affect mouse exploratory and spatial working memory in both mice types.

### Example 4

### Spatial Learning and Memory in Mice Fed on Dunaliella Algae Powder Preparation

The spatial learning and memory were next measured by the Barnes maze. WT mice are expected to have short latency period compared to the Alzheimer's-like mouse model (Tg2576). As shown in FIG. 4, preliminary results (n=6-10) from this on-going experiment showed that the latency period is longer in Tg2576 control mice in comparison to WT mice on day 1 and day 7 after the training period. The shorter latency period in the Dunaliella algae powder preparation group shows that Dunaliella algae powder preparation diet improved cognitive function in Tg2576 mice; on day 7 the improvement was statistically significant (p<0.05), and on day 1 there was a trend toward shorter latency. Interestingly, a trend toward a lower latency period was also detected in the WT Duna. Prep. group compared to WT control group.

The above results indicates that Dunaliella algae powder preparation improved long term memory, as demonstrated by the Tg2576 mice treated with the Duna. Prep. of the invention.

### Example 5

### 9-Cis β-Carotene Crossed the Blood-Brain Barrier

In order to reach the brain, β-carotene has to cross the blood brain barrier (BBB). Once consumed in the food, β-carotene has been previously reported to be carried in the blood stream by chylomicrons and low density lipoprotein (LDL).

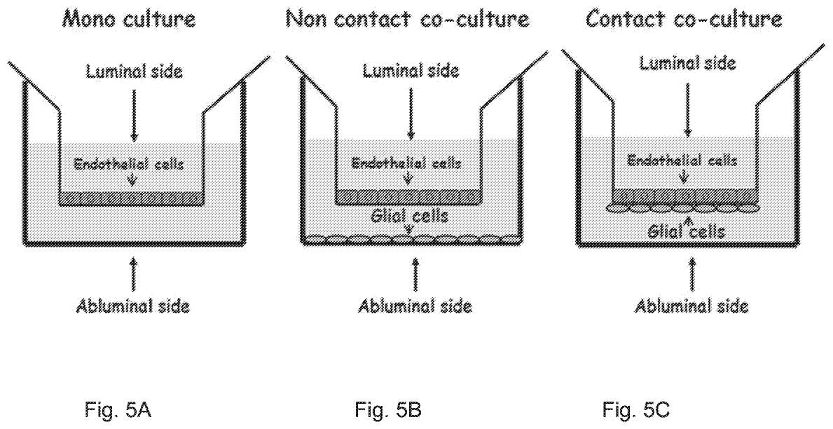

Using the “BBB in-vitro model” described above and schematically shown in FIG. 5A, 5B, 5C, glial and endothelial cells were incubated for 24 hours in the presence of LDL (inherently containing all-trans and 9-cis β-carotene) and then samples were collected and carotenoids were extracted and analyzed using HPLC.

As shown in FIG. 6B, 9-cis β Carotene as well as all-trans β Carotene crossed the BBB. As shown in FIG. 6A, lycopene, on the other hand, apparently did not cross the BBB, suggesting the selectivity of the model.

In addition, the “BBB in-vitro model” described above is also used for assessment of crossing the BBB by carotenes in LDL extracted from a healthy volunteer that are administered with the Dunaliella algae diet described herein.

### Example 6

### Dunaliella's 9-Cis β Carotene Crossed the Blood Brain Barrier and Accumulated in Brain and Peripheral Tissues

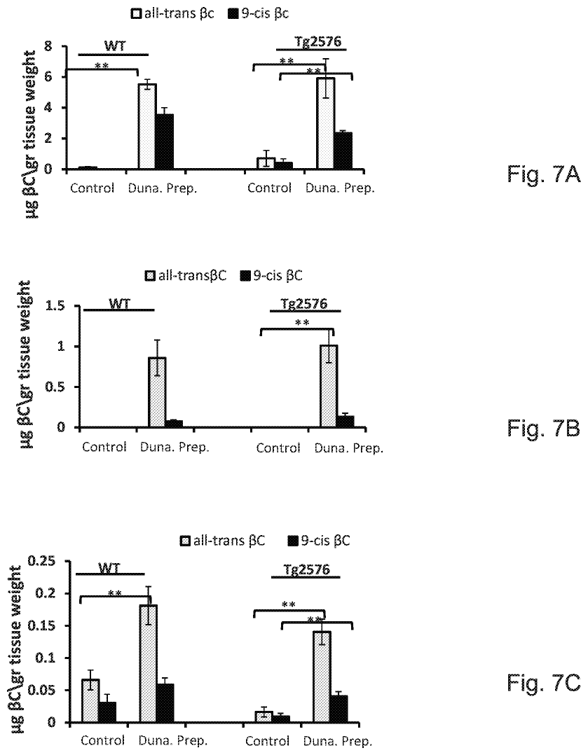

In order to assess whether carotenoid level was higher in brain tissue of Tg2576 mice fed with the Dunaliella diet as compared to the control group, mice brain carotenoids were extracted and then measured using HPLC. As shown in FIG. 7, while in the control diet there were negligible tissue levels of 9-cis β Carotene, in mice fed with the Dunaliella diet there were much higher levels of both all-trans and 9-cis β Carotene. In particular, in the brain of the group fed on Dunaliella diet, the carotenoid level was significantly higher than in the control groups (FIG. 7C). The results suggest that exposure to a diet rich in carotenoids lead to its accumulation in body tissues, specifically, liver (FIG. 7A), fat (FIG. 7B), including the brain (FIG. 7C), and thus may be a source for brain carotenoids which in turn may be a source for brain retinoids.

### Example 7

### Insoluble and Soluble Amyloid β Levels in Tg2576 Mice Fed on Dunaliella Algae Powder Preparation

One of the main histological features of AD is neurotic plaques (amyloid beta, β-amyloids). As detailed above, it has been previously suggested that retinol and β-carotene potentially inhibit amyloid β formation. The Inventors next examined the effect of the Dunaliella algae powder preparation diet on Aß peptides levels (insoluble and soluble) in the hippocampus of the assayed Tg2576 mouse. For quantitative assessment of hippocampal formation of Aß peptides, total Aß1-42 was measured by sandwich ELISA as detailed above.

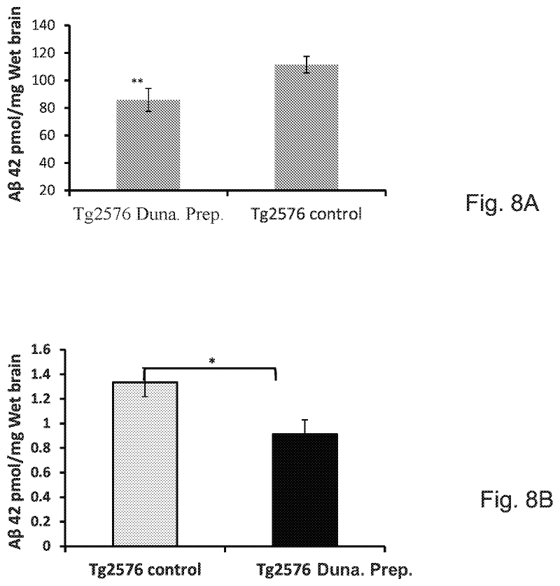

As demonstrated in FIG. 8A, the insoluble β-amyloid levels were significantly (p=0.02) reduced in Tg2576 mice fed with Dunaliella algae powder preparation (Tg2576 Duna. Prep.) over the control group (fed on regular diet). A similar effect was demonstrated for the soluble β-amyloid fraction, as shown in FIG. 8B.

### Example 8

### Analysis of Gene Expression in Tg2S76 Mice Fed on Dunaliella Algae Powder Preparation Diet

Without wishing to be bound by theory, the treatment described herein may affect Alzheimer's disease via three possible (hypothetical) pathways: via ACB transporters and lipids, BBB, plasticity and inflammation.

Therefore, expression of RXR and downstream genes, namely apoE, ABCA1 and ABCG1 were assessed in the hippocampus of animals fed on the Dunaliella algae powder preparation rich diet or control diet, and in addition, expression of the above genes was assayed in vitro, in hippocampal neurons culture. Total RNA was extracted and analyzed by RT-βCR. Analysis of protein expression was measured by quantitative Western blotting.

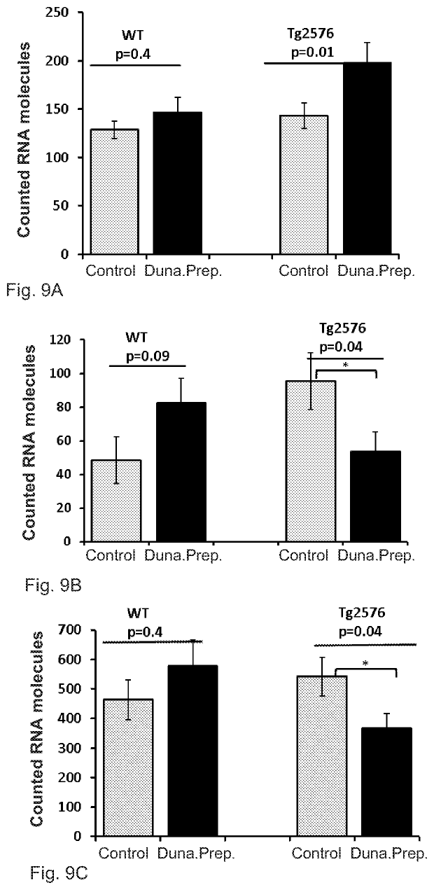

Specifically, a NanoString gene expression analysis was applied on the hippocampus of Tg2576 mice participating in the above detailed assay. Only gene expression values that met the threshold criteria specified in the Methods section above were considered to be statistically significant. Gene expression levels that were significantly affected by the treatment detailed herein were: Il-1α (ratio 1.4, P value=0.01) (FIG. 9A), Il-1β (ratio 0.54, P value=0.04) (FIG. 9B) and TSPO (ratio 0.69, P value=0.04) (FIG. 9C). All three are inflammatory genes. The results suggest a possible beneficial effect of for the Dunaliella algae powder preparation diet on neuroinflammation in the context of AD, as shown in FIG. 9. Further genes involved in inflammation for which a higher expression levels was shown are IL-6 (about 1.3) and MCP1 (about 1.5).

### Example 9

### TSPO Protein Level

Following the gene expression results above demonstrating that the positive effects observed for the Dunaliella algae diet may involve inflammatory genes, the effect of the Dunaliella algae powder preparation was examined on TSPO protein expression, whose upregulation is considered to be a hallmark of microglial activation. In order to evaluate the TSPO protein level, the protein extract of Tg2576 mice hippocampus was quantified using western blot analysis (FIG. 10). Normalized blots with tubulin showed a significant decrease (p=0.01) in TSPO level in treated mice, 25% decrease compared to Tg2576 control mice, suggesting an anti-inflammatory effect to the Dunaliella algae powder preparation.

### Example 10

Cholesterol and Triglyceride Levels in the Plasma of Mice Fed with the Dunaliella Algae Powder Preparation

In order to study the effect of the Dunaliella algae powder preparation on plasma cholesterol and triglycerides in the Tg2576 mice model animals, fasting plasma lipid levels were measured. As shown in FIG. 11, the Dunaliella diet significantly (p=0.02) lowered plasma cholesterol levels in treated WT mice but did not affect triglyceride levels therein. In contrast, in the Tg2576 treated mice there was no effect on cholesterol level, however the Dunaliella diet had an effect on triglyceride levels (p=0.07).

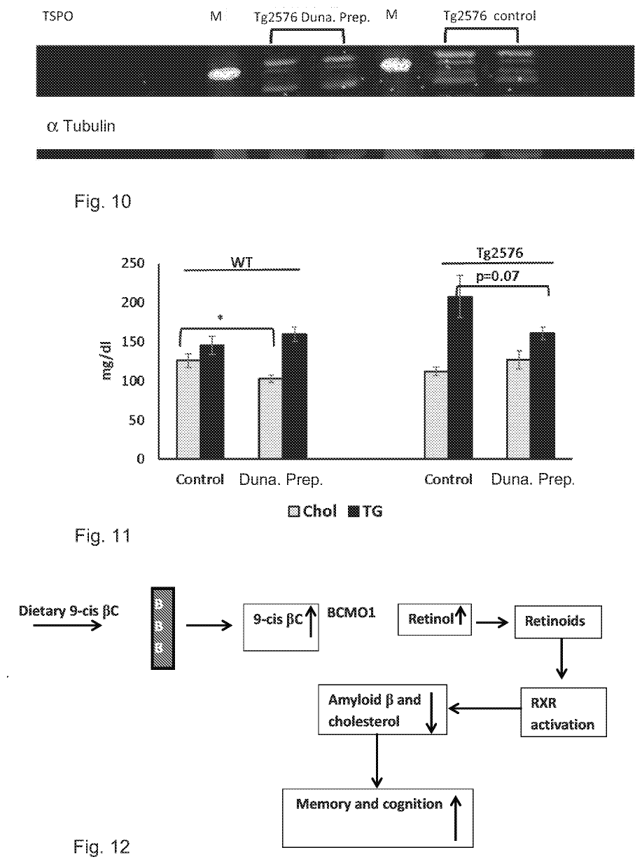

Taken together, the above results indicate that dietary 9-cis β-carotene can cross the BBB and may be converted to retinoids within the brain cells by enzymatic activity (e.g. of the enzyme BCMO1). A schematic diagram showing a proposed mechanism of action of dietary 9-cis β-carotene is shown in FIG. 12. Without wishing to be bound by theory, increased levels of retinoids intensify clearance of Aβ and consequently improve cognitive function. The Dunaliella algae preparation described herein may be available rich source for 9-cis β-carotene and other isomers thereof or substances.

### Example 11

### β-Carotene in the Brain and Peripheral Tissues of 5×FAD Mice Fed on the Dunaliella Algae Powder Preparation

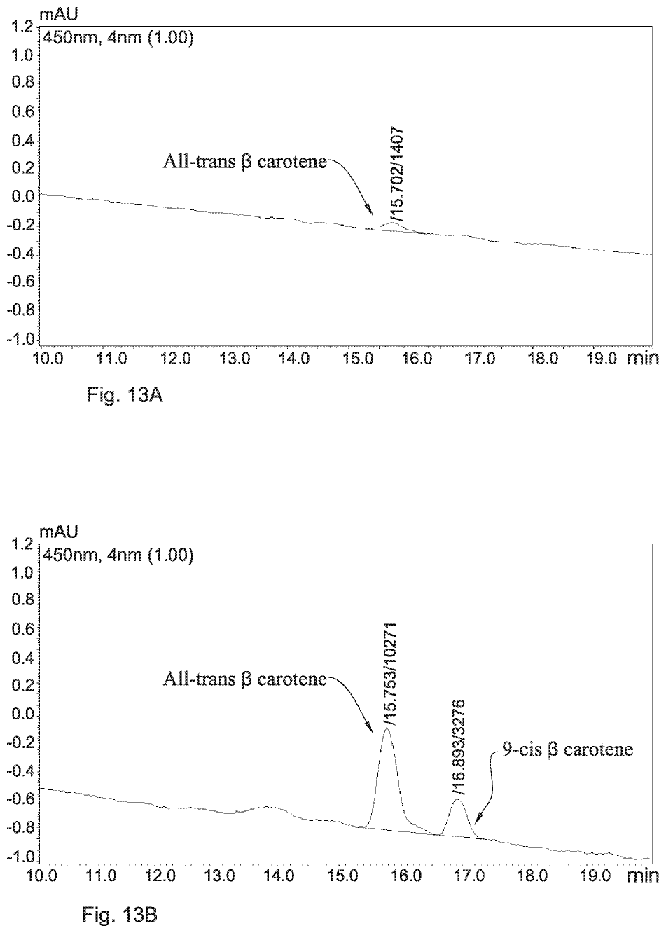

Next, the effect of the Dunaliella algae powder preparation was examined on the 5×FAD mice model. In a first assay, performed on brain extracts of 5×FAD mice fed on the Dunaliella algae powder preparation, it was shown that 9-cis β Carotene and all-trans β Carotene crossed the BBB, as demonstrated in FIG. 13. As shown in FIG. 13B, brain tissues of mice fed on the Dunaliella algae diet as described above contained significantly higher amounts of 9-cis β Carotene and all-trans β Carotene as compared to the level of the above β Carotene isomers in brain tissues of mice fed on a regular diet (FIG. 13A).

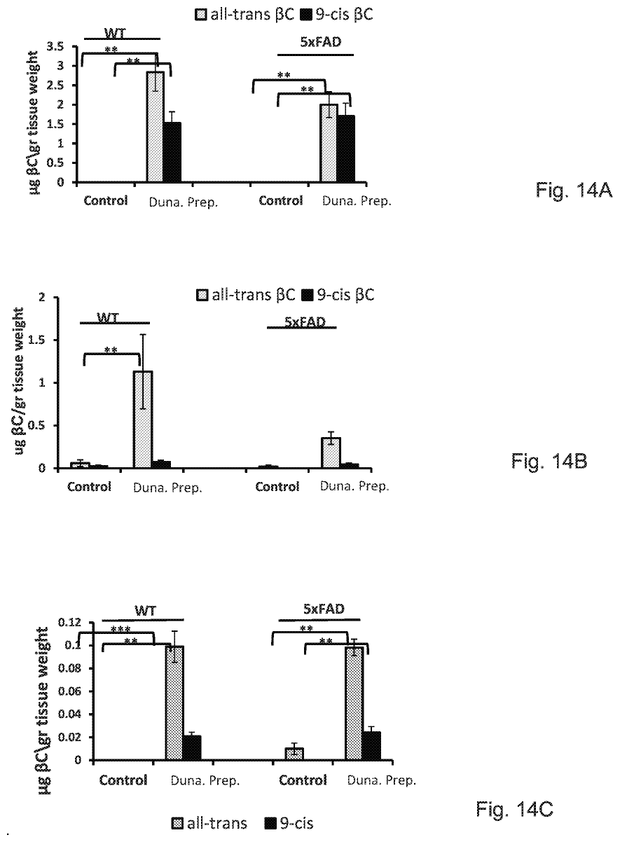

In addition and similarly to the experiment performed for the Tg2576 model, carotenoids were shown to cross the BBB and accumulate in tissues at higher levels in the Dunaliella algae powder preparation fed animals. As shown in FIG. 14, these results suggest that all-trans and 9-cis β Carotene originating from the alga Dunaliella crossed the BBB and accumulated in liver, fat and brain tissues (FIGS. 14A, 14B, 14C, respectively) at much higher levels in the Dunaliella diet group compared to the control group. In addition, carotenoid levels in this model were similar to those in the Tg2576 mice model.

Without wishing to be bound by theory, since β-Carotene crosses the BBB as demonstrated both in vitro and in vivo, it can be a source for local production of retinoids in the brain.

### Example 12

### Cholesterol and Triglyceride Levels in the Plasma of 5×FAD Mice Fed on the Dunaliella Diet

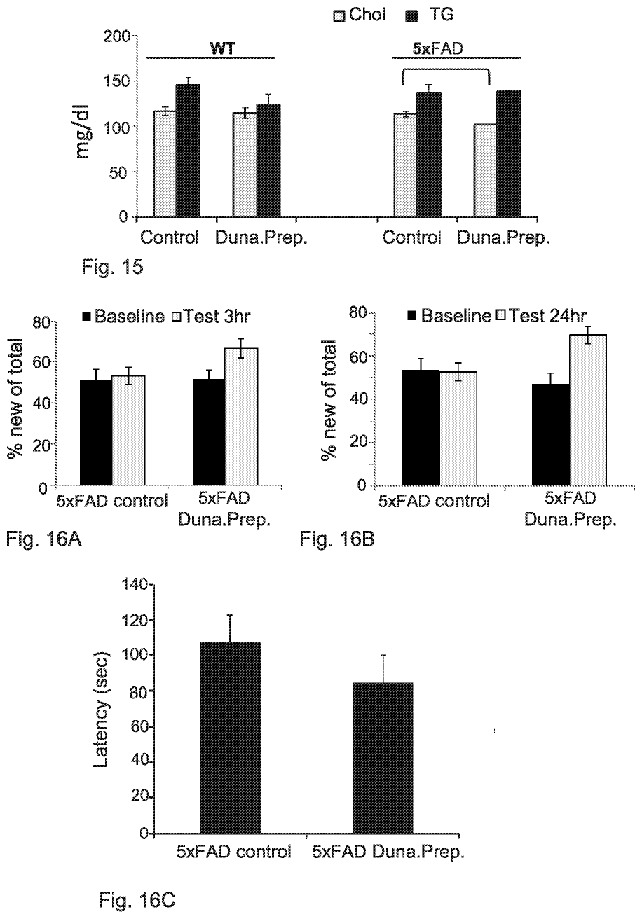

In order to study the effect of the Dunaliella diet on plasma cholesterol and triglycerides, fasting plasma lipid levels were measured as described in the methods section above. As shown in FIG. 15, the Dunaliella diet significantly (p=0.04) lowered plasma cholesterol levels in the Dunaliella diet fed 5×FAD mice but did not affect triglyceride levels in these animals. In WT mice, the Dunaliella diet did not affect neither cholesterol nor triglycerides.

### Example 13

### Novel Object Recognition in 5×FAD Mice Fed on Dunaliella Algae Powder Preparation

In the test described herein, the mouse is presented with two identical objects for 5 minutes, and 3 hours later (for short term memory) and 24 hours late one of the objects is replaced by a different one (for long term memory). The length of time taken to explore the new object provides an index of recognition memory. This test was performed in 5×FAD mice fed on Dunaliella algae powder preparation, as described above, aiming to explore the effect of the Dunaliella diet on memory.

As demonstrated in FIG. 16A, the Dunaliella algae powder preparation diet improved short term memory in 5×FAD mice as deduced from the higher percentage of new objection recognition in the 5×FAD mice fed on the Dunaliella algae powder preparation diet (5×FAD Duna. Prep.).

In addition, as shown in FIG. 16B, the Dunaliella algae powder preparation diet also improved long term memory in 5×FAD mice.

As demonstrated in FIG. 16C, 5×FAD mice fed on the Dunaliella algae powder preparation (Duna. Prep.) diet showed a positive trend (P=0.051) toward improved long term memory.

### Example 14

### The Effect of Dunaliella Algae Powder Preparation on Spatial Working Memory

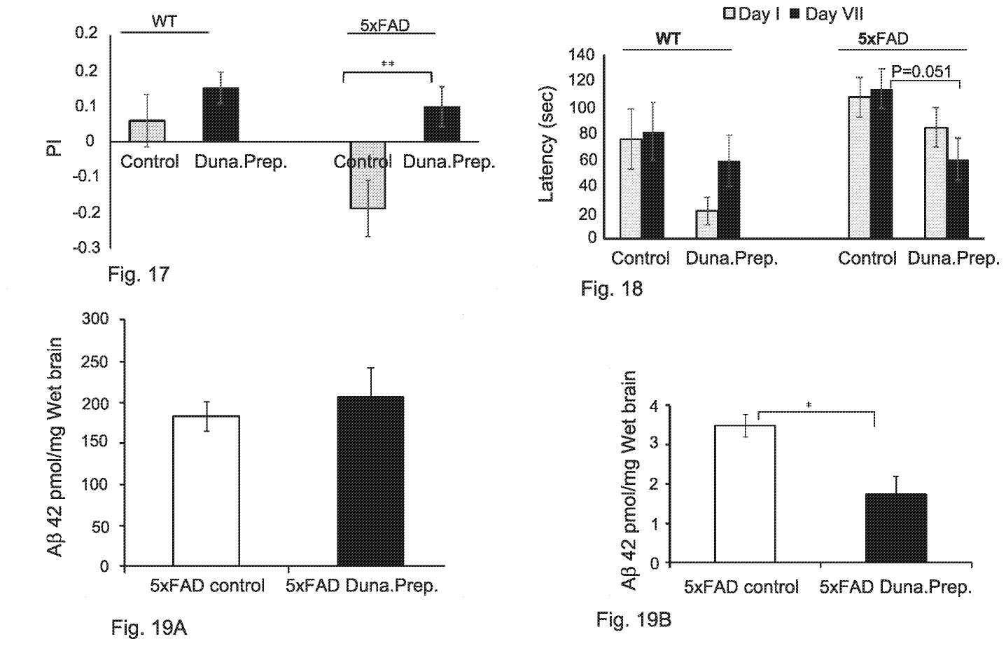

In order to examine the spatial working memory of the 5×FAD mice, the Y-maze test was used and the mice innate preference to explore novel areas was measured. Mice with normal cognition are expected to spend more time in the new arm. As shown in FIG. 17, 5×FAD mice that were fed on the Dunaliella algae powder preparation showed significantly (p=0.009) improved short-term memory (mean PI=0.09±0.05) compared to the control group (mean PI=−0.18±0.07).

### Example 15

### The Effect of Dunaliella Algae Powder Preparation on Long-Term Memory

In order to examine mice long-term memory, the Barnes maze test was used, as elaborated in the methods section above. As shown in FIG. 18, the latency period is longer in 5×FAD control mouse in comparison to 5×FAD fed with the Dunaliella algae powder preparation both on day 1 and on day 7 of the experiment. Mice fed with the Dunaliella algae powder preparation (61 seconds±16) showed a positive trend (p=0.051) toward improved long-term memory as compared to control 5×FAD group (114 seconds±15) on the second recall test in day 7 of the experiment.

### Example 16

### The Effect of Dunaliella Algae Powder Preparation on the Levels of Amyloid β in Mice Brain Hippocampus

Amyloid § was extracted from 5×FAD mice hippocampus as described in the method section above, in order to evaluate the level thereof and to examine the possible effect of the Dunaliella algae powder preparation. The results shown in FIG. 19 demonstrate that although there is no effect for the Dunaliella algae powder preparation on insoluble Aβ (p=0.5), as shown in FIG. 19A, there was a significant reduction (p=0.03) in soluble Aβ level in mice receiving 9CBC (1.7±0.2) compared to control mice (3.4±0.4), as shown in FIG. 19B.

1. A method for preventing, treating, ameliorating, reducing or delaying the onset of at least one of a neurodegenerative disease, a disorder associated with protein misfolding, cognitive decline and any conditions or symptoms associated therewith in a subject in need thereof, comprising administering to said subject an effective amount of at least one Dunaliella algae preparation or any composition comprising thereof.

2. The method according to claim 1, wherein said method results in amelioration or reduction of at least one condition or symptom associated with at least one of a neurodegenerative disease, a disorder associated with protein misfolding and cognitive decline in a subject in need thereof.

3. The method according to claim 2, wherein said condition or symptom is at least one of short term memory impairment, long term memory impairment, impaired cognitive function, impaired learning function, β-amyloids deposition, anxiety, depression, or any combination thereof.

4. The method according to claim 1, wherein said method results in improvement of at least one of cognitive function, short term memory, long term memory, acquisition time and clearance of β-amyloids in a subject in need thereof.

5. The method according to claim 1, wherein said neurodegenerative disease is at least one of Alzheimer's disease, Parkinson's disease, Mild Cognitive Impairment (MCI), Parkinson's disease with MCI, Huntington's disease, Lewy body disease, Amyotrophic lateral sclerosis (ALS), Prion disease, Motor neuron disease (MND), Spinocerebellar ataxia (SCA), Spinal muscular atrophy (SMA), Friedreich's Ataxia and any other neurodegenerative-related dementia or ataxia.

6. The method according to claim 5, wherein said neurodegenerative disease is Alzheimer's disease.

7. The method according to claim 1, for preventing, treating, ameliorating, reducing or delaying the onset of cognitive decline.

8. The method according to claim 1, wherein said Dunaliella algae is Dunaliella bardawil.

9. The method according to claim 1, wherein said Dunaliella algae preparation is administered orally.

10-18. (canceled)

19. A method for improving at least one of short term memory impairment, long term memory impairment, impaired cognitive function, impaired learning function, β-amyloids deposition in a subject in need thereof, comprising administering to said subject an effective amount of at least one Dunaliella algae preparation or any composition comprising thereof.

20. The method according to claim 19, wherein said Dunaliella algae preparation or any composition thereof improve at least one of cognitive function, short term memory, long term memory, acquisition time and clearance of β-amyloids in a subject suffering from a neurodegenerative disease.

21. The method according to claim 19, wherein said neurodegenerative disease is at least one of Alzheimer's disease, Parkinson's disease, Mild Cognitive Impairment (MCI), Parkinson's disease with MCI, Huntington's disease, Lewy body disease, Amyotrophic lateral sclerosis (ALS), Prion disease, Motor neuron disease (MND), Spinocerebellar ataxia (SCA), Spinal muscular atrophy (SMA), Friedreich's Ataxia and any other neurodegenerative-related dementia or ataxia.

22. The method according to claim 21, wherein said neurodegenerative disease is Alzheimer's disease.

23. The method according to claim 19, wherein said Dunaliella algae is Dunaliella bardawil.

24. The method according to claim 19, wherein said Dunaliella algae preparation is administered orally.

25-34. (canceled)
